(19) United States
(12) Patent Application Publication
(10) Pub. No.: US 2018/0149639 A1
(43) Pub. Date: May 31, 2018

(54) MODELING NEURAL NETWORK DYSFUNCTION

(71) Applicants: The Broad Institute, Inc., Cambridge, MA (US); Massachusetts Institute of Technology, Cambridge, MA (US)

(72) Inventors: Jen Q. Pan, Acton, MA (US); Congyi Lu, Allston, MA (US); Guoping Feng, Newton, MA (US)

(21) Appl. No.: 15/526,625

(22) PCT Filed: Nov. 13, 2015
(86) PCT No.: PCT/US2015/060703

§ 371 Date: May 12, 2017

Related U.S. Application Data
(60) Provisional application No. 62/080,039, filed on Nov. 14, 2014.

(51) Int. Cl. G01N 33/50; C12N 5/0793

(52) U.S. Cl. CPC G01N 33/5058; C12N 2502/081; G01N 2800/28; C12N 5/0619

(57) ABSTRACT

The present invention relates to in vitro models of autism and methods of using the same to diagnose disorders associated with network dysfunction, e.g., autism schizophrenia, depression, obsessive-compulsive spectrum disorders, bipolar disorder, or epilepsy, and to identify compounds for use in treating these conditions.

### CROSS-REFERENCE TO RELATED APPLICATIONS

This application claims the benefit of U.S. Application No. 62/080,039, filed on Nov. 14, 2014. The entire contents of the foregoing are incorporated by reference herein.

### TECHNICAL FIELD

The present invention relates to in vitro models of autism and methods of using the same to diagnose disorders associated with network dysfunction, e.g., autism spectrum disorders, schizophrenia, schizoaffective disorder, depression, obsessive-compulsive spectrum disorders, bipolar disorder, or epilepsy, and to identify compounds for use in treating those conditions.

### BACKGROUND

Autism is a neurodevelopmental disorder that afflicts 1 in 88 children in the United States and 1% of all human population (Baird et al., 2006). The etiology of autism remains elusive, yet autism has a strong heritable genetic susceptibility (Precht et al., 1998; Wilson et al., 2003). Several studies have shown that deletions and mutations of the Shank3 gene play a causative role in Phelan-McDermid syndrome (PMDS), an autism spectrum disorder (ASD) (Gauthier et al., 2009; Durand et al., 2007; Boeckers et al., 2002). The Shank3 gene encodes a postsynaptic scaffolding protein critical for the development and function of excitatory synapses in the brain (Shcheglovitov et al., 2013; Baron et al., 2006; Hayashi et al., 2009; Sheng & Kim, 2000), by recruiting important signaling molecules and neurotransmitter receptors to the postsynaptic density. Deleting exons 13-16 in the PDZ domain of Shank3 eliminated both the short and long isoforms of Shank3. The resulting animal, known as Shank3B KO, robustly captured two cardinal phenotypes of autism (social interaction deficits and repetitive behavior) and was found with reduced number of synapses in cortico-striatal connection, as well as reduced cortico-striatal excitatory synaptic transmission (Peca et al., 2011). Thus, neurons from Shank3B KO mice were used in this report as the Shank3 KO autism model in culture. However, it is still unclear whether losing shank3 during development has a specific impact on the cortical network output, hyper-excitatory or hypo-excitatory, or whether the absence of shank3 protein influences the balance between inhibitory and excitatory input at the network level.

### SUMMARY

The present invention is based, at least in part, on the discovery that optimized primary neuronal cultures and micro-electrode array (MEA) detection can be used together to create an in vitro model of the cellular/electric signature of disorders associated with network dysfunction, e.g., autism spectrum disorders, schizophrenia, schizoaffective disorder, depression, obsessive-compulsive spectrum disorders, bipolar disorder, and epilepsy. This model can be used to screen drugs against this “signature” to identify compounds that could correct defects associated with these conditions, or for diagnosis or identification of optimal therapy for personalized treatment of individuals with these conditions.

Thus, in a first aspect, the invention provides in vitro methods for identifying a candidate compound for the treatment of a condition associated with neural network dysfunction. The methods include providing an in vitro model of the condition, wherein the model comprises a co-culture comprising a network of inhibitory and excitatory neurons; detecting a first level of network activity in the model; contacting the model with a test compound; detecting a second level of network activity in the model in the presence of the test compound; comparing the second level of network activity to the first level of network activity; and selecting a test compound that is associated with an altered, e.g., decreased, level of network activity, as a candidate compound.

In some embodiments, the first level of network activity is a baseline level of network activity, or a level of network activity in the presence of a stimulus that increases activity in the network.

In a further aspect, the invention provides in vitro methods for diagnosing the presence of a condition associated with neural network dysfunction in a subject. The methods include providing an in vitro model of the condition, wherein the model comprises a co-culture comprising a network of inhibitory and excitatory neurons, wherein the neurons are obtained by a method comprising differentiating stem cells, e.g., iPSCs (induced pluripotent cells) or neural progenitor cells, e.g., made from somatic cells, of a subject suspected of having the condition; detecting a first level of network activity in the model; contacting the model with a stimulus that increases activity in the network; detecting a second level of network activity in the model in the presence of the stimulus; comparing the second level of network activity to the first level of network activity; assigning a subject value to the difference between the first and second levels of network activity; comparing the subject value to a reference value, wherein the reference value represents a level of activity in the presence of the stimulus in a subject who does not have a condition associated with neural network dysfunction; and identifying a subject as having a condition associated with neural network dysfunction based on the presence of a subject value above the reference value.

In yet another aspect, the invention provides in vitro methods for selecting a treatment for a condition associated with neural network dysfunction in a subject. The methods include providing an in vitro model of the condition, wherein the model comprises a co-culture comprising a network of inhibitory and excitatory neurons, wherein the neurons are obtained by a method comprising differentiating stem cells, e.g., iPSC or neural progenitor cells, e.g., made from somatic cells, of a subject suspected of having the condition; detecting a baseline level of network activity in the model; contacting the model with a stimulus that increases activity in the network; detecting a stimulated level of network activity in the model in the presence of the stimulus; contacting the model with a test compound, e.g., a pharmaceutical treatment for the condition; detecting a baseline level of network activity in the model in the absence of the test compound, and a stimulated level of network activity in the model in the presence of the test compound; comparing the baseline and stimulated levels of network activity in the presence of the test compound; assigning a first value to the difference between the baseline and stimulated levels of network activity in the presence of the test compound; comparing the baseline and stimulated levels of network activity in the absence of the test compound; assigning a second value to the difference between the first and second levels of network activity in the absence of the test compound; comparing the first and second values, to detect a level of change in the values; comparing the change in the values to a reference level of change, wherein the reference level of change represents a level associated with a positive control compound, e.g., a successful treatment, that reduces network activity in the presence of the stimulus; and selecting a test compound that causes a level of change that is equal to or greater than the reference level of change.

In some embodiments, the stimulus is administration of a chemical agent (e.g., GABA receptor agonists, antagonists and modulators including GABA-A receptor agonists (e.g., muscimol), antagonists (e.g., bicuculline and picrotoxin (PTX)) or allosteric modulators (e.g., benzodiazepines and Flumazenil); GABA-B receptor agonists (e.g., baclofen), antagonists (e.g., saclofen) or allosteric modulators (e.g., CGP-7930); glutamic acid decarboxylase inhibitors (e.g., Semicarbazide); Type I mGluR agonists/positive modulators (e.g., 3,5-dihydroxyphenylglycine (DHPG)) and antagonists/negative modulators (e.g., MPEP); calcium channel agonists (e.g. BAY K8644) and antagonists (e.g., Nifedipine); BDNF (brain derived neurotrophic factors); or neuromodulators such as dopamine, acetylcholine, and serotonin), or electrical stimuli, e.g., an electrical pulse, e.g., square depolarization of about 1-4 ms, e.g., 2 ms, at various frequencies (for example, 10 or 100 Hz).

In some embodiments, the inhibitory neurons are GABAergic neurons, and the excitatory neurons are glutamatergic neurons.

In some embodiments, the neurons, e.g., one or both of the inhibitory neurons and the excitatory neurons, are primary neurons obtained from the brain of an animal, e.g., a cadaver or an animal model of the condition.

In some embodiments, the inhibitory neurons are obtained from the striatum of the brain, and/or the excitatory neurons are obtained from the cortex of the brain. In some embodiments, the culture comprises, or is substantially purely or enriched in (e.g., at least 80%, e.g., at least 85%, 90%, or 95%) neurons from the cortex, hippocampus, thalamus, or striatum, or a mixture of neurons from the cortex and striatum (i.e., cortico-striatal coculture), thalamus and cortex (i.e., thalamo-cortical coculture), or cortex and hippocampus (i.e., cortico-hippocampal coculture).

In some embodiments, the neurons are obtained by a method comprising differentiating stem cells or neural progenitor cells, e.g., stem or neural progenitor cells obtained from a subject having the condition or made from cells from a subject having the condition, e.g., from iPS cells made from somatic cells of a subject, e.g., a subject having the condition.

In some embodiments, the neurons are obtained by a method comprising differentiating stem cells or neural progenitor cells, e.g., ES cells or iPS cells made from somatic cells, of a subject having the condition, and the method further comprises administering the selected test compound to the subject. In some embodiments, the neurons are wild type; in some embodiments, the neurons have been genetically manipulated to comprise a mutation associated with a condition as described herein. For example, the neurons may be differentiated from a human or animal ES or iPS cell that has been genetically engineered to harbor a mutant gene associated with a condition as described herein, e.g., SHANK3 or islet brain-2 mutants for autism.

In some embodiments, detecting a level of network activity comprises detecting one or more of types of bursts, bursting durations, spike rate within bursts, the number of spikes in each burst; and frequency of bursts, and/or ratios thereof, e.g., ratios of bursting durations and inter-burst interval durations.

In some embodiments, comparing the second level of network activity to the first level of network activity comprises determining the power spectrum for each level, calculating the area under the curve (AUC) of specific frequency domains in the power spectrum, and comparing the AUC, or comparing the area over a specific frequency range.

In some embodiments, comparing the second level of network activity to the first level of network activity comprises determining the network oscillation by one or both of autocorrelogram or crosscorrelogram analysis for each level, calculating the area under the curve (AUC) in each frequencies, and comparing the AUC, or comparing the area over a specific frequency range.

In some embodiments, the condition associated with neural network dysfunction is autism spectrum disorders, schizophrenia, schizoaffective disorder, depression, obsessive-compulsive spectrum disorders, bipolar disorder, or epilepsy, or Phelan-McDermid Syndrome (PMS).

In another aspect, the invention provides compositions comprising a co-culture comprising a network of inhibitory and excitatory neurons, wherein the composition models a neural network dysfunction, e.g., autism spectrum disorders, schizophrenia, schizoaffective disorder, depression, obsessive-compulsive spectrum disorders, bipolar disorder, Phelan-McDermid Syndrome (PMS), and epilepsy.

In some embodiments, the inhibitory neurons are GABAergic neurons, and the excitatory neurons are glutamatergic neurons. In some embodiments, the neurons are primary neurons obtained from the brain of an animal. In some embodiments, the inhibitory neurons are obtained from the striatum of the brain, and/or the excitatory neurons are obtained from the cortex of the brain.

In some embodiments, the neurons are obtained by a method comprising differentiating stem cells or neural progenitor cells, e.g., ES cells or iPS cells made from somatic cells, of a subject having or suspected of having the dysfunction.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F: Spontaneous firings of cortical neurons in vitro revealed by MEA.

(A) Threshold-based spike detection for collecting time-stamps for MEA data analysis. (Left) Phase-contrast microphotograph of cortical neurons on MEA at DIV21; (Right) Recorded signals were high-pass filtered at 200 Hz to remove the low-frequency local field potentials. Threshold-based spike detection was used to identify and collect timestamps for MEA data analysis. (B-C) Representative raster plots show spontaneous firing activities recorded from one WT cortical culture (B) and one Shank3 KO cortical culture (C) at various developmental ages. (D-F) Simultaneous GCaMP imaging and MEA recording from cortical neurons on MEA. (D) Representative images show neurons on MEA infected with AAV2/8-CaMKII-GCaMP6F. Left: fluorescent image of GCaMP6F; Middle: phase-contrast image from the same filed to indicate the location of recording electrodes; Right: overlapped fluorescent and phase-contrast image. (E-F) Calcium transients captured from GCaMP imaging (top) are shown aligned in time with raster plots simultaneously recorded from MEA (bottom) from WT culture (E) and Shank3 KO culture (F). Each GCaMP trace represents the fluorescence signals detected from the soma of one cell body.

FIGS. 2A-F: Shank3 KO cortical cultures exhibit reduced spike rate in both spontaneous and PTX-treated network.

(A) Representative traces of MEA recording from WT and Shank3 KO cortical cultures at DIV21. (B) Left, quantification of mean spike rate from MEA recordings of WT and Shank3 KO cultures at DIV14, 18 and 21. Right, cumulative distribution of mean spike rate from WT and Shank3 KO cultures at DIV21. Data derived from 54 MEAs for WT group and 68 MEAs for Shank3 KO group from 6 independent batches of littermate cultures. $$$, p<0.001 compared to DIV14 within the same genotype, unpaired t test; *, p<0.05, **, p<0.01, compared to WT at the same DIV, unpaired t test. (C) Comparison of mean spike rate from WT vs. Shank3 KO cultures at DIV 21 (left) and the fold change of mean spike rate for Shank3 KO cortical cultures to enhancing excitatory synaptic transmission by AMPA receptor positive modulator CX546 (right). Data for WT and Shank3 KO comparison was the same used in panel B; Data for effect of CX546 on Shank3 KO derived from 6 MEAs from 2 independent batches of cultures. **, p<0.01, compared to WT, unpaired t test; #, p<0.05, ##, p<0.01, compared to Shank3 KO before treatment (Basal), paired t test. (D) Representative traces of MEA recording from WT and Shank3 KO cortical cultures at DIV21 treated with 50 μM PTX. (E) Mean spike rate analysis of WT and Shank3 KO cultures at DIV21 before and after PTX (50 μM) treatment. Data derived from 21 WT and 21 Shank3 KO MEAs from 5 independent batches of litter-mate cultures. ###, p<0.001, compared to the same genotype before treatment, paired t test; **, p<0.01, compared to WT of the same condition, unpaired t test. (F) Comparison of mean spike rate from PTX-treated WT vs. Shank3 KO cultures at DIV 21 (left) and the fold change of mean spike rate for PTX-treated Shank3 KO cortical cultures to enhancing excitatory synaptic transmission by AMPA receptor positive modulator CX546 (right). Data derived from 8 MEAs from 2 independent batches cultures. **, p<0.01, compared to WT, unpaired t test; #, p<0.05, ##, p<0.01, compared to Shank3 KO with 50 μM PTX only (basal), paired t test. For all results, data are mean±SEM.

FIGS. 3A-C: Shank3 KO cortical cultures exhibit altered network firing pattern.

(A) ASDR plot used for analyzing network firing pattern was generated by summing the number of spikes detected per 200 ms over all electrodes and graphed over time. As marked on the top of representative ASDR plot, red/green bars illustrate network active period (NAP) and network inactive period (NIP) to indicate the slow network oscillation. Inset was a zoomed-in view showing ASDR spike and inter-spike interval (ISI). NAP, NIP, ASDR spike and ISI was captured by threshold-based waveform collection as explained in detail in method. (B) Representative ASDR plots show network active period (NAP) and network inactive period (NIP) alterations for WT and Shank3 KO cortical cultures at DIV21. (C) Quantification of the duration of NAP and NIP from WT and Shank3 KO networks at DIV21. Cumulative plot and average values (inset) were shown in each panel. Data were from 40 ASDRs for WT and 38 ASDRs for Shank3 KO from 30 minutes MEA recordings of 5 independent batches of litter-mate cultures. **, p<0.01, compared to WT, unpaired t test.

FIGS. 4A-D: Response of WT in vitro cortical networks to pharmacological manipulation of excitatory/inhibitory synaptic transmission.

Representative ASDR plots (top) and the analysis of fold change in the duration of NAP and NIP (bottom) for WT cortical networks at DIV21 in response to (A) GABA_{A} receptor positive modulator clonazepam (CLP); (B) GABA_{A} receptor antagonist picrotoxin (PTX); (C) AMPA receptor positive modulator CX546; (D) AMPA receptor antagonist NBQX. Data derived from >4 MEAs in each experimental group from 2 independent batches of cultures. Data are mean±SEM. *, p<0.05, **, p<0.01, compared to before treatment (Basal), paired t test.

FIGS. 5A-B: The effect of CX546 and clonazepam on the duration of NIP in Shank3 KO networks

(A) Left: ASDR plots from a representative Shank3 KO network in response to AMPA receptor positive modulator CX546; Right: the analysis of NIP duration for WT networks and Shank3 KO networks before and after the treatment of CX546. Data derived from 3 MEAs from 1 batch of litter-mate cultures. One-way ANOVA, F(4,85)=11.14, p<0.0001; ** p<0.01, *** p<0.001 compared to WT. (B) Left: ASDR plots from a representative Shank3 KO network in response to GABA_{A} receptor positive modulator clonazepam (CLP); Right: the analysis of NIP duration for WT networks and Shank3 KO networks before and after the treatment of CLP. Data derived from 8 MEAs in each experimental group from 2 independent batches of litter-mate cultures. One-way ANOVA, F(4,210)=11.07, p<0.0001; ** p<0.01, nsp>0.05 compared to WT. For all results, data are mean±SEM.

FIG. 6: Spike rate analysis of WT cortical neurons on MEA with development.

Spike rate analysis of cortical cultures at 5 distinct ages. Data derived from 18 MEAs for each age group from 3 independent culture preparations. Data are presented as mean±SEM; one-way ANOVA, F(4,1216)=41.4, p<0.0001; *** p<0.0001 compared to DIV7.

FIGS. 7A-B: Spontaneous firings of DIV21 Shank3KO network without NAP/NIP alternation.

Representative raster plots (A) and ASDR plot (B) show spontaneous firing activities recorded from one DIV21 Shank3 KO culture without NAP/NIP alternation.

FIGS. 8A-B: Comparison of bursting activities from WT and Shank3 KO cortical cultures with development.

(A) Illustration of the bursts detecting using NeuroExplorer 4 software. Bursts were defined as spike activities occurred with maximal interval to start burst of 10 ms, maximal interval to end burst of 100 ms, minimal interval between bursts of 100 ms, minimal duration of burst of 50 ms, and minimal number of spikes in burst of 5. (B) Quantitative analysis of burst rate, burst duration, number of spikes in burst and spike rate in burst from WT and KO cultures at DIV14, 18 and 21. Data derived from 54 MEAs for WT group and 68 MEAs for Shank3 KO group from 6 independent batches of littermate cultures. $$$, p<0.001 compared to DIV14 within the same genotype, unpaired t test; *, p<0.05, **, p<0.01, compared to WT at the same DIV, unpaired t test.

FIGS. 9A-B: Characterization of Shank3 protein expression level for cortical neurons in vitro.

(A) Representative western blots of Shank3 and (3-actin using whole cell lysates from cortical neurons at three distinct developmental ages in vitro. (B) Quantification of Shank3 expression level relative to β-actin in cortical neurons in culture collected at DIV 7, 14, and 21. Data derived from 3 independent batches of cultures. Data are mean±SEM. One-way ANOVA, F(3,8)=8.79, p=0.0165; *, p<0.05, compared to DIV7.

FIGS. 10A-B: Quantification of total neurons and GABAergic neurons in wild type and Shank3 KO cortical cultures.

(A) Representative images of WT cortical culture immunostained for GABA (red), NeurN (green) and DAPI (blue). (B) Quantification of NeuN positive cells and GABA positive cells from WT and Shank3 KO cultures at DIV21. Data derived from 3 independent batches of litter-mate WT and Shank3 KO cultures at the density comparable to those used in MEA recordings (3×10³ cells/mm²). Data are mean±SEM. ns, p>0.05 compared to WT, unpaired t test.

FIGS. 11A-F: Change in the duration for NAP upon pharmacological manipulation of excitatory/inhibitory synaptic transmission.

(A-D) Quantitative analysis of fold change in the duration of NAP for WT cortical networks at DIV21 in response to GABA_{A} receptor positive modulator clonazepam (CLP) (A), GABA_{A} receptor antagonist picrotoxin (PTX) (B), AMPA receptor positive modulator CX546 (C), and AMPA receptor antagonist NBQX (D). Data derived from ≥4 MEAs in each experimental group from 2 independent batches of cultures. (E-F) Quantitative analysis of fold change in the duration of NAP for Shank3 KO cortical networks at DIV21 in response to AMPA receptor positive modulator CX546 (E) and GABA_{A} receptor positive modulator clonazepam (CLP) (F). Data derived from >3 MEAs in each experimental group from >1 independent batches of cultures. For all results, data are mean±SEM. *, p<0.05, **, p<0.01, compared to before treatment (Basal), paired t test.

### DETAILED DESCRIPTION

While a rational target-based drug discovery paradigm has successfully led to novel, effective therapeutic agents for breast cancer (Her2-neu blocker Lapatinib) or lung cancer (EGFR inhibitor Tarceva), this approach has not served well in most disorders of the nervous system. The optimal molecular targets for biological circuits or pathways for treating Autism spectrum disorders (ASD), schizophrenia (SCZ) or other neurological disorders such as Huntington's are largely unknown, and the molecular mechanisms underlying these disorders are not understood, necessitating phenotypic (cell-based) screens for the discovery of drug candidates. In addition, targets implicated by genetics in nervous system disorders are often among protein classes generally considered “undruggable” (e.g., scaffolding proteins, transcription factors). Therefore, cell-based assays aimed at relevant molecular pathways involving the target(s) represent a possible therapeutic screening strategy in psychiatric disorders. Unfortunately, few complex cellular phenotypes underlying major psychiatric disorders are known. Furthermore, traditional functional assays for synaptic function (LTP, LTD, patch clamp, synaptic transmission) and dendritic morphology (spine morphology, synaptogenesis) are not yet amenable for high throughput. Clearly, new and more powerful approaches to cell-based screens for neurological disorders are urgently needed.

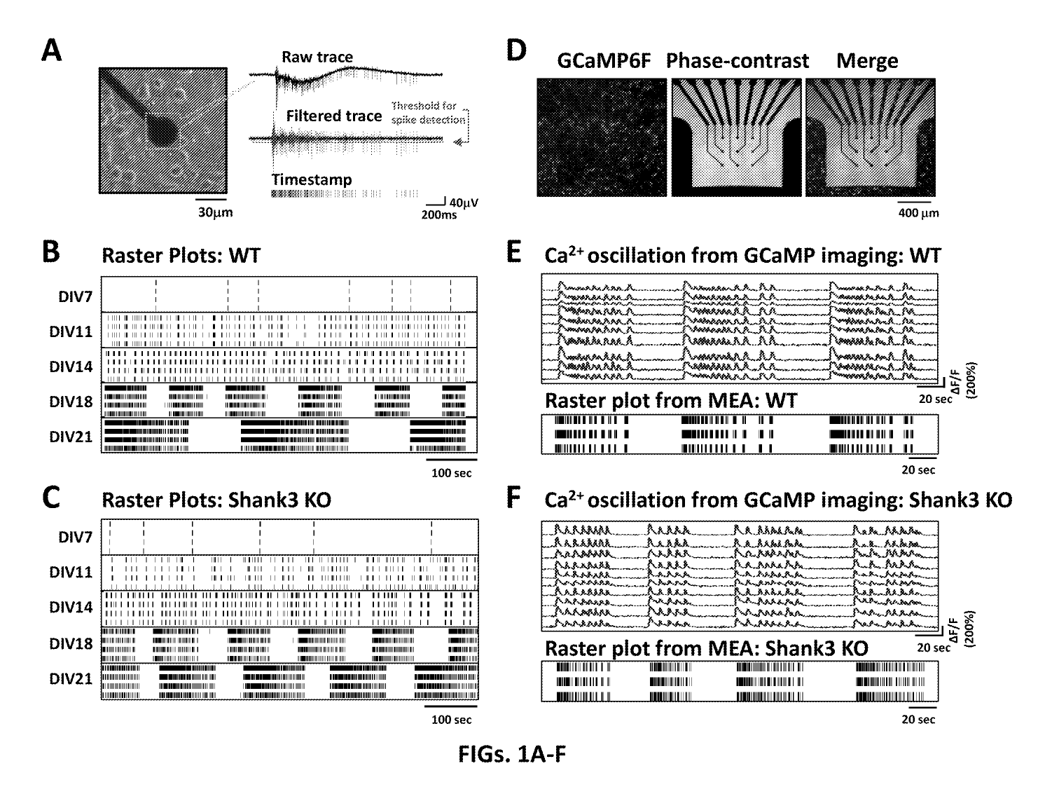

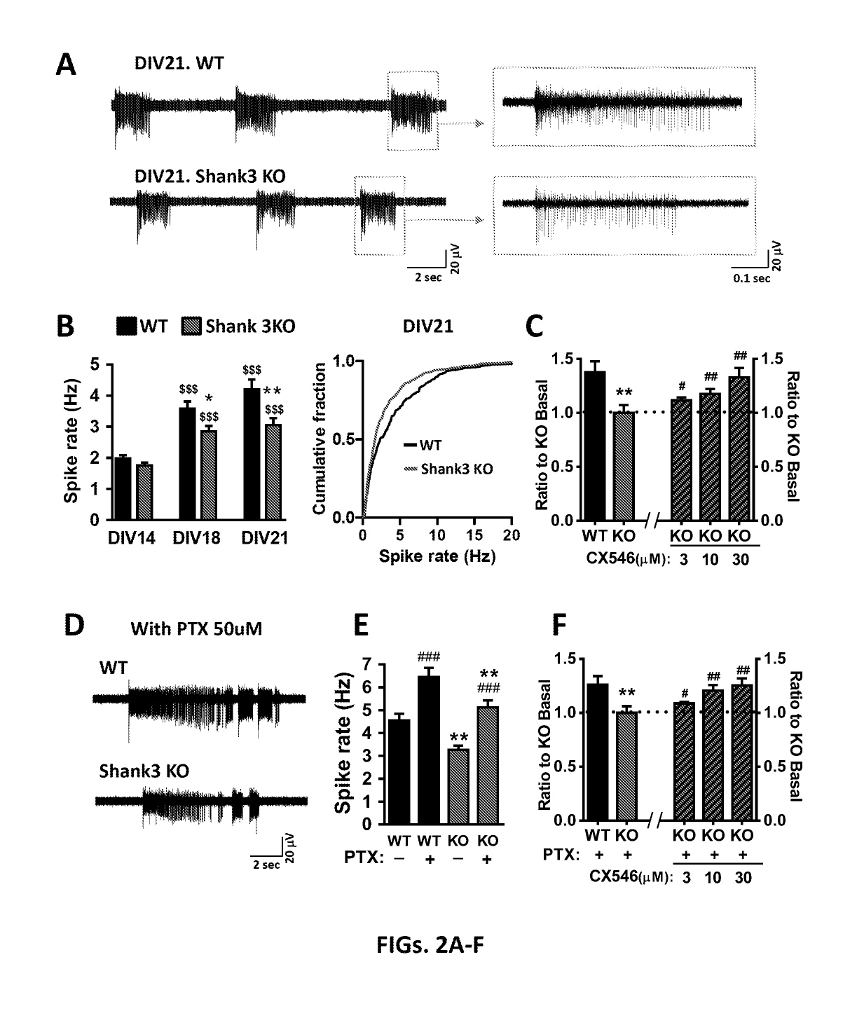

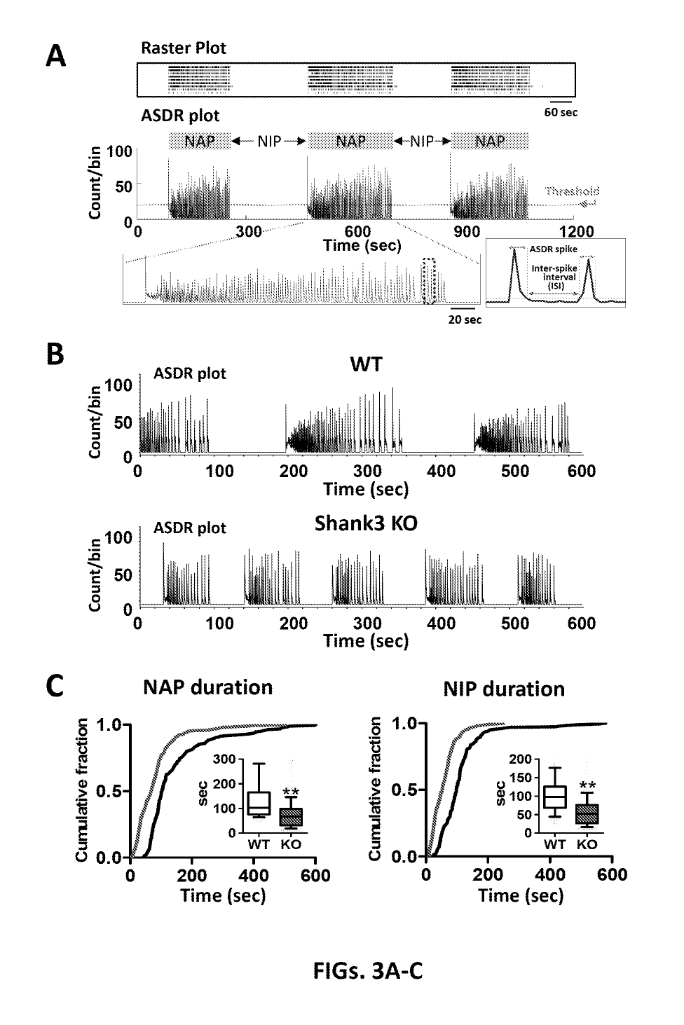

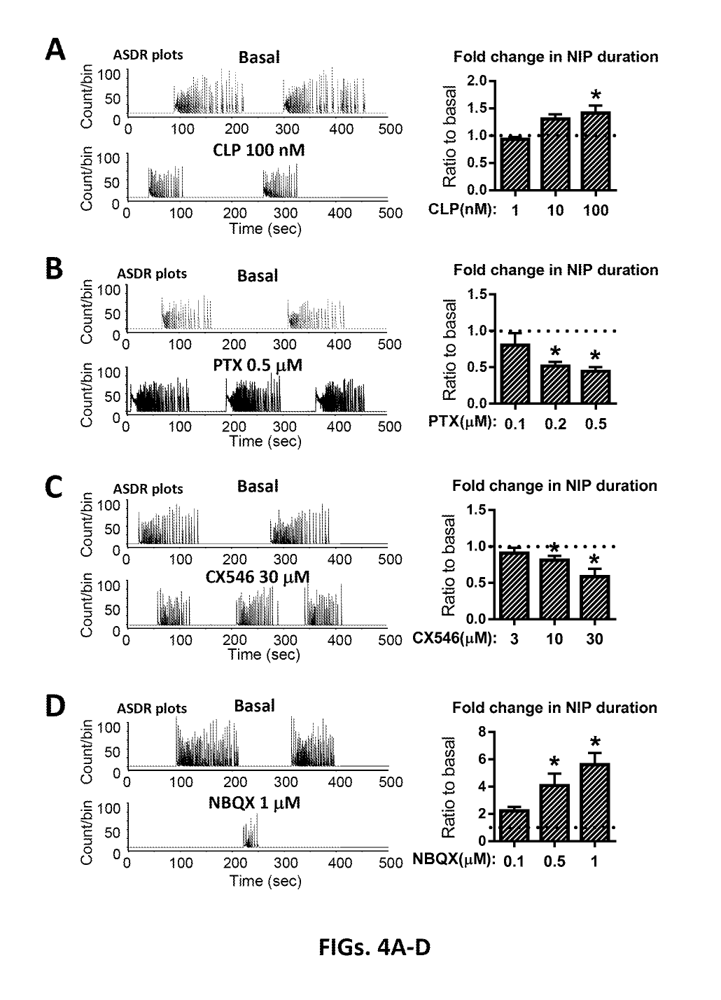

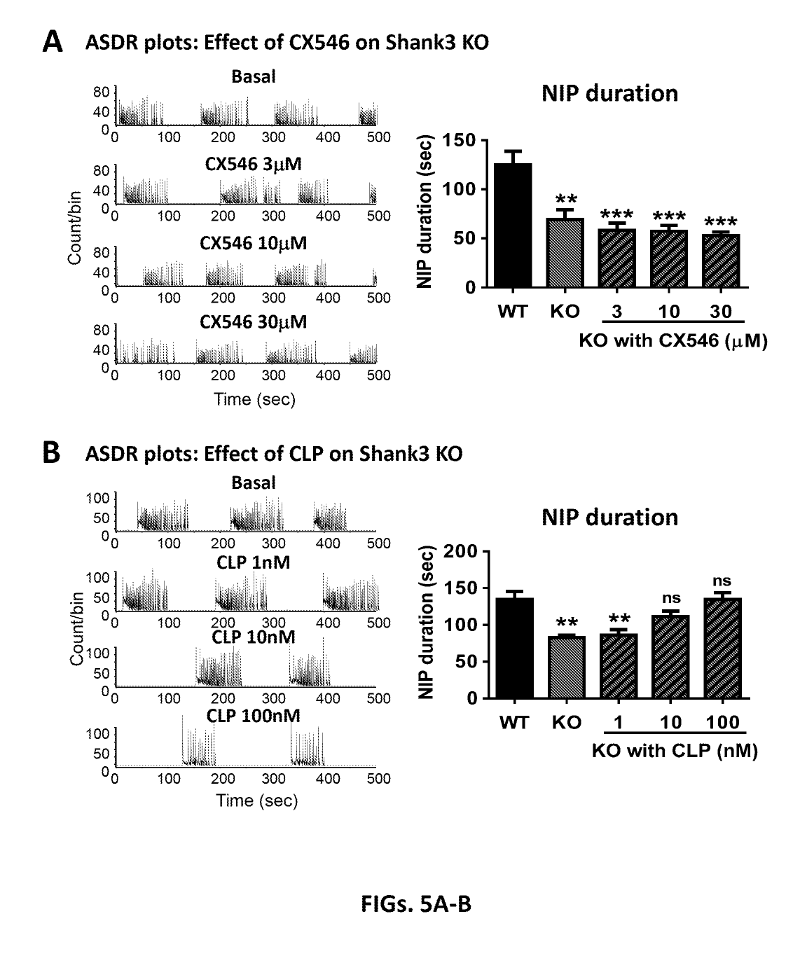

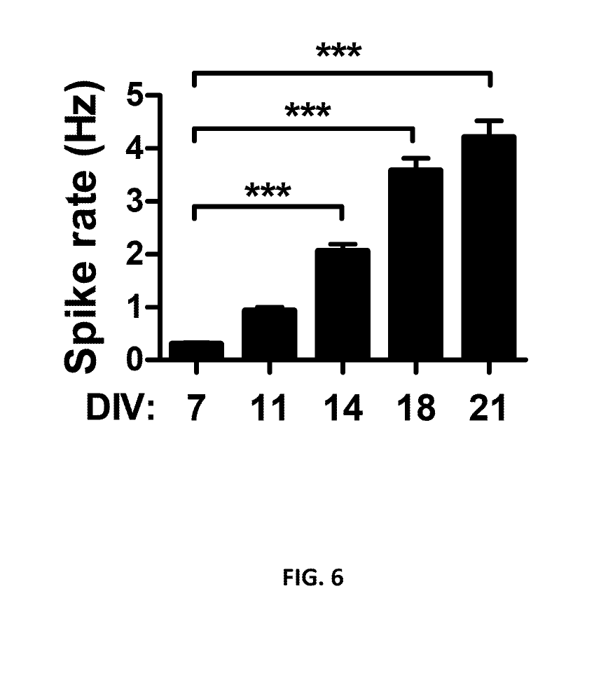

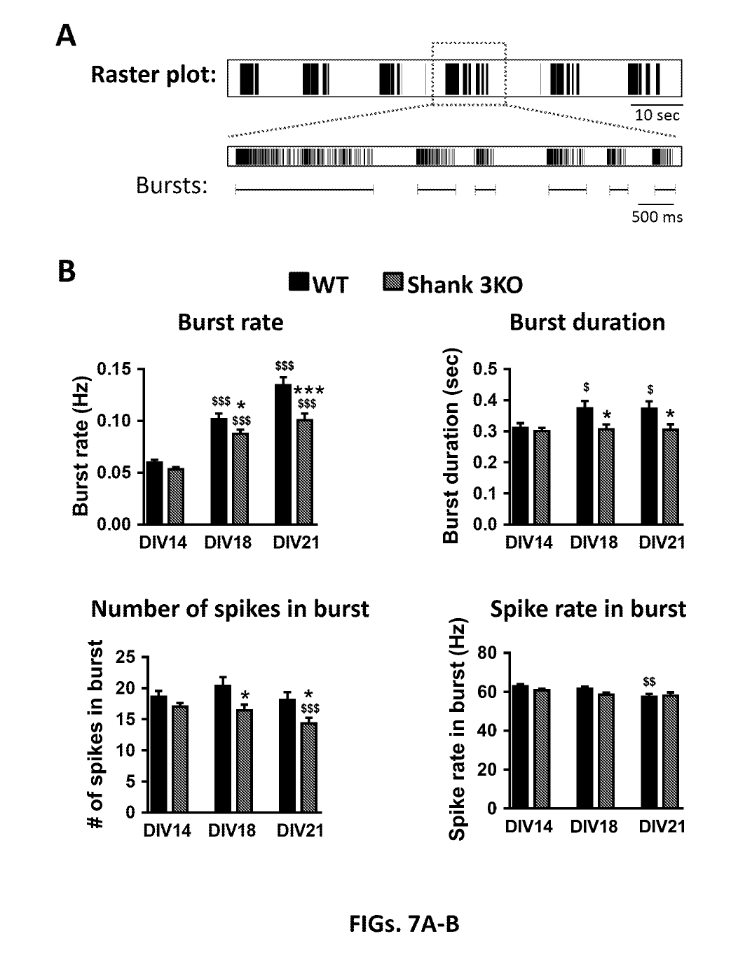

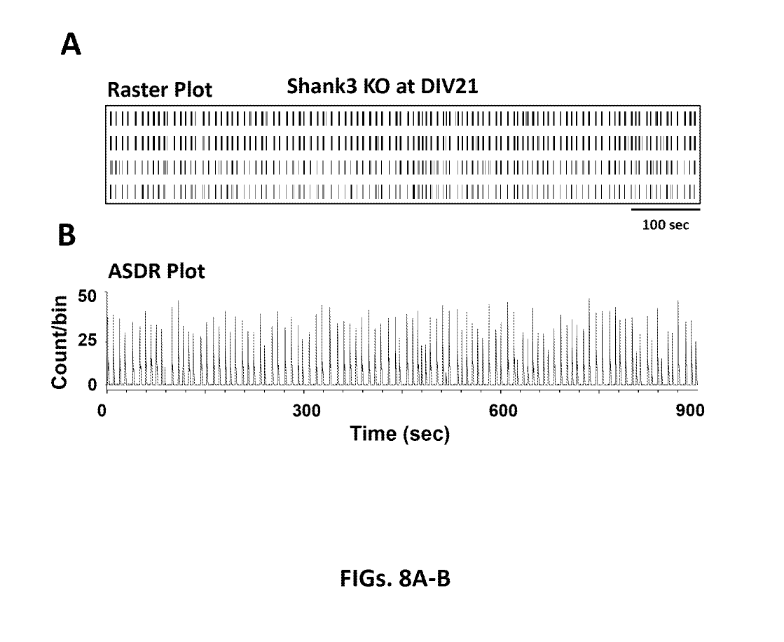

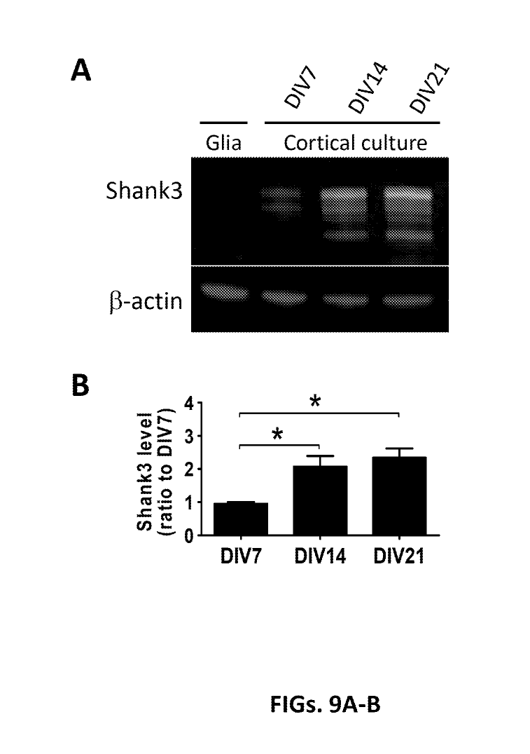

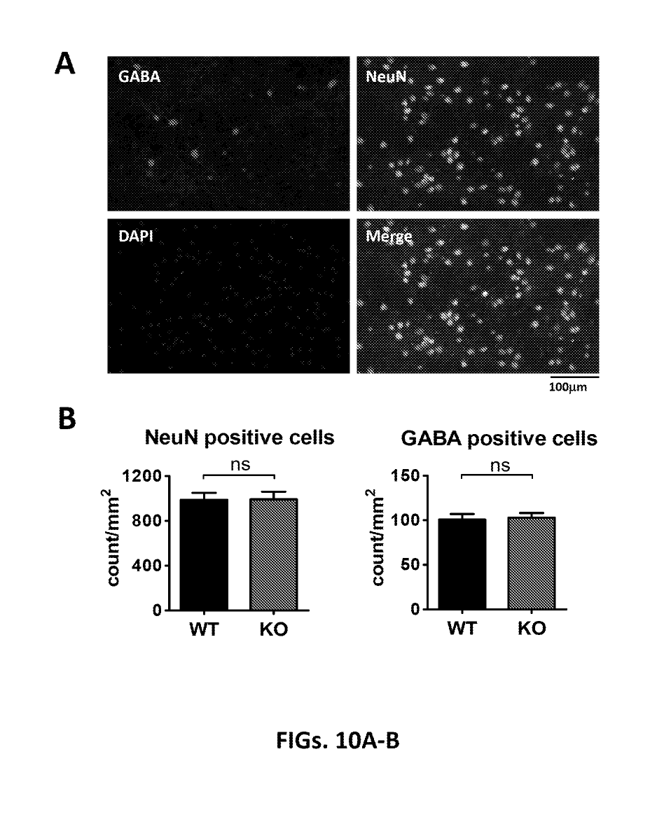

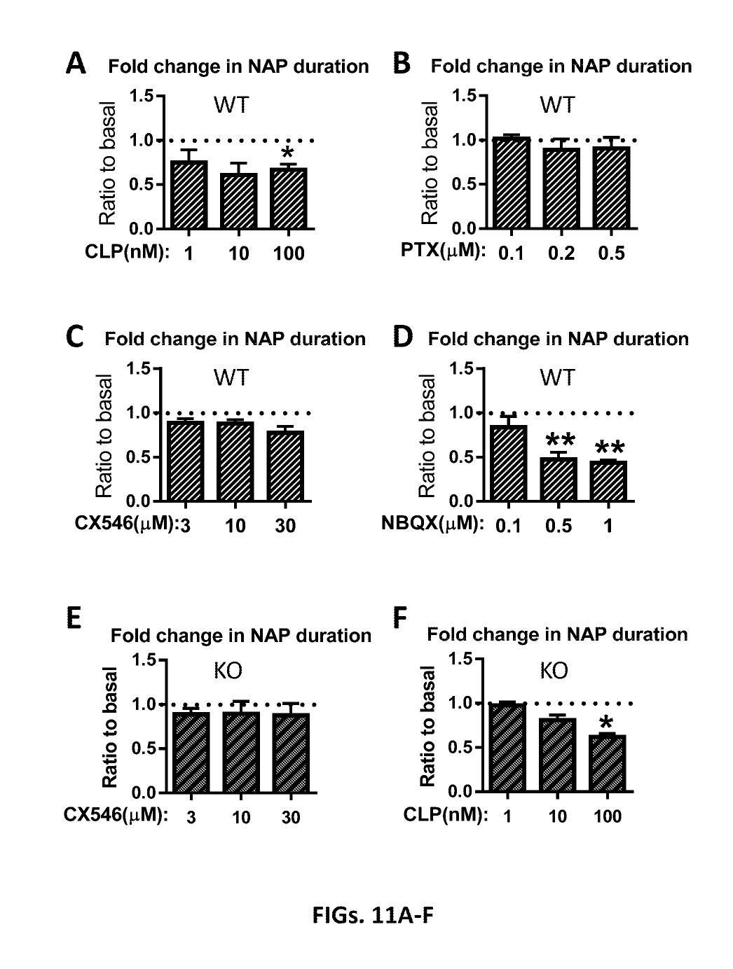

Using the MEA recordings of high-density cortical cultures, we analyzed the salient features of dynamic neural networks. We found that cortical networks in vitro form robustly reproducible active (NAP) and quiet (NIP) bi-stable states (FIGS. 1 and 3). While network-spiking frequency measures the overall network output (FIGS. 6 and 7), the duration of inactive periods of the network (NIP) reflect the relative excitatory and inhibitory strength in the network (FIG. 4). Subsequently, we found that cultured Shank3 KO neurons showed a reduction in spontaneous firing rate and shorter NIPs (FIGS. 2 and 3). The reduced spontaneous firing rates was rescued by glutamatergic transmission enhancer CX546 (30 μM, FIG. 2C), and the shortened NIP duration in Shank3 KO network was normalized by GABA_{A} receptor positive modulator clonazepam (100 nM, FIG. 5B). These results, coupled with our pharmacological profiling of in vitro cortical networks (FIG. 4) in the presence of synaptic transmission modulators, suggest concurrent reduction of excitatory and inhibitory synaptic function in the network lacking Shank3 protein, and an imbalance between excitatory and inhibitory neuronal input to the network contributes to the change in network oscillation pattern.

### Dynamic Network Oscillations Reflect the Balance Between Excitatory and Inhibitory Input to the Network

Previous reports have cataloged diverse forms of network patterns in rat cortical cultures with MEA study (Wagenaar et al., 2006). We optimized culture conditions for growing cortical neurons from P0 mice on MEA, and found that our cultures generated reproducible network firing patterns featuring prolonged, alternate periods of active (NAP) and inactive (NIP) network activities lasting for tens of seconds developed at or after DIV18 (FIG. 1). Alternating excited and quiet bi-stable states of the network have been reported in both in vitro and in vivo cortical circuits (Mok et al., 2012; Aladjalova, 1957). However, no studies have investigated the biological significance for such very slow network oscillation. A theoretical framework for bi-stable states network has been described by Wilson and Cowan (1972) in a simple model that consists of a population of excitatory and inhibitory neurons with mutual connection and self-connection at a much faster time scale. The excited and quiet states of the Wilson and Cowan model are determined by a balance between the mutual excitation among excitatory neurons and the feedback inhibition they generate by the way of inhibitory population. Consistent with the Wilson and Cowan model, it has been shown that when hippocampal neurons, composed of mainly pyramidal neurons, cultured with increasing amount of inhibitory neurons, producing longer quiet state (NIP) within the network (Chen et al., 2010), suggesting that the amount of inhibitory feedback to the network regulates the structure of network quiescence. Using the method we developed for quantifying the NAP and NIP, we found that pharmacological manipulation of glutamatergic and GABAergic synapses induces changes in the NAP/NIP cycling structure (FIG. 4). Specifically, increasing inhibitory synaptic transmission (FIG. 4A) or reducing the excitatory synaptic transmission (FIG. 4D) dose dependently increased NIP duration. Conversely, decreasing inhibitory control of the network (FIG. 4B), or increasing excitatory input to the network (FIG. 4C) dose dependently reduced the duration of NIP. Our results show, for the first time, that the structure of NAP/NIP oscillation reflects the relative strength of both excitatory and inhibitory synaptic transmission. Interestingly, NAP is less sensitive to the modulation of glutamatergic or GABAergic synaptic transmission compared to NIP, suggesting that NIP duration is more sensitive to the change in the balance between excitation and inhibition.

### Impaired Excitatory and Inhibitory Synaptic Transmission in the Shank3 KO Networks

Using MEA recordings, we found that cortical Shank3 KO neurons generated networks with reduced spiking frequency (FIG. 2B), and CX546, an AMPA receptor positive modulator, rescued the hypoactive network in Shank3 KO network. Coupled with the fact that Shank3 is a postsynaptic protein, our data indicate that reduced glutamatergic synaptic function may underlie the hypoactive firing properties in Shank3 KO network. We also demonstrated that Shank3 KO neurons still fired less in the treated network upon PTX application to remove all inhibitory input to the network (FIGS. 2E and 2F), confirming that the excitatory transmission is attenuated in the Shank3B KO network. These results are consistent with the previous reports that Shank3 deletion caused defects in excitatory synaptic transmission in Shank3 KO models (Peca et al., 2011; Shcheglovitov et al., 2013).

More strikingly, we found that Shank3 KO cortical networks cycle faster between NAP and NIP bi-states, with significantly reduced NIP duration (48.5% reduction, FIG. 3B). Together with our result that reduced inhibition or increased excitation generate a network with shorter NIP duration (FIG. 4), our data suggest that neurons in the absence of Shank3 produce attenuated inhibitory feedback in the network. More importantly, CLP normalized reduced NIP duration in Shank3 KO network by enhancing GABAergic function (FIG. 5B). In comparison, CX546, while rescuing the hypoactive firing in Shank3 KO network, did not recover the network defect (FIG. 5A). Altogether, our experimental results indicate that reduced excitatory transmission in the absence of the Shank3 protein led to overall network hypoactivity, and, that lacking Shank3 protein also impaired inhibitory feedback input to the network and such attenuation led to altered network oscillation patterns including reduced duration for NIP.

Our results on the impaired inhibitory function in cortical Shank3 KO network are corroborated by the recent report that Shank3 KO mice display altered inhibitory neuron markers including decreased intensity, number and size of PV puncta per pyramidal cell soma, suggesting an immature or weakened inhibitory circuitry in the insular cortex (Gogolla et al., 2014). Given that many autism and neurodevelopmental disorder models have pointed to defects in inhibitory neurons function (Rubenstein et al., 2003; Gogolla et al., 2009; Chao et al., 2010), it may be plausible that normalizing reduced inhibitory feedback input to the Shank3B network may normalize the defect at the organism and behavioral level. Future investigations including testing neurons from other autism models (TSC, MeCP, or FRMP) using dense culture in vitro network will address whether altered NIP/NAP network oscillation structures exist as a common cellular phenotype across multiple different neurodevelopmental and autism models.

### Therapeutic Discovery

We demonstrated that MEA recordings could monitor spontaneous and treated network patterns, locally and globally, that are sensitive to glutamatergic and GABAergic synaptic function in vitro. Phenotyping neuronal functions with traditional measurements of synaptic activities (synaptic transmission, LTP, LTD) with patch-clamp or dendritic morphology (spine morphology, synaptogenesis) are difficult to scale up (Sharma et al., 2013; Hempel et al., 2011). MEA is amenable for higher throughput non-invasive recordings of intact networks in vitro, and a 48-well MEA system is available (Valdivia et al., 2014) for simultaneous recording. We have demonstrated that MEA recording not only measures the output of the network (spike rate), but also reads the NIP/NAP network structure that reflect the input from excitation/inhibition of the network. Furthermore, we identified specific electric MEA signatures in the Shank3 loss of function model network. Our results indicate that MEA is a powerful, unbiased functional assay for intact neural networks. Recent GWAS implicated 108 genetic loci that are associated for the risk for schizophrenia (Ripke et al., 2014), and exome studies implicated many genes to be associated with autism and schizophrenia (Iossifov et al., 2014). The present results show that we can phenotype and categorize these genetic hits associated with disease risk by their MEA electro-phenotypes to identify converging molecular, cellular, and even network pathways that underlie the emerging genetics of mental illnesses.

### In Vitro Models of Neural Network Dysfunction

Described herein are in vitro models that replicate the neural defects seen in patients suffering from conditions associated with neural network dysfunction. A number of psychiatric and neurological conditions are associated with neural network dysfunction, e.g., autism spectrum disorders (including Phelan-McDermid Syndrome (PMS)), schizophrenia, and epilepsy. See, e.g., Uhlhaas and Singer, “Neural Synchrony in Brain Disorders: Relevance for Cognitive Dysfunctions and Pathophysiology” Neuron 52(1):155-168, 2006; Gordon and Moore, “Charting a course toward an understanding of schizophrenia” Neuron. 76(3):465-7, 2012; Welch et al., “Cortico-striatal synaptic defects and OCD-like behaviors in Sapap3-mutant mice,” Nature. 448(7156):894-900, 2007; Mayberg, “Targeted electrode-based modulation of neural circuits for depression,” J Clin Invest. 119(4):717-25, 2009; Lisman et al., “Circuit-based framework for understanding neurotransmitter and risk gene interactions in schizophrenia,” Trends Neurosci. 31(5):234-42, 2008.

In preferred embodiments, the model includes co-cultures comprised of both inhibitory (i.e., GABAergic (produce the neurotransmitter GABA), and/or DARPP32+ neurons) and excitatory (i.e., glutamatergic (produce the excitatory neurotransmitter glutamate) CamKII+ neurons) neurons, preferably in a ratio ranging from 1:1 to 1:3 of excitatory to inhibitory, e.g., of cortical:striatal neurons. In some embodiments, the culture comprises, or is substantially purely (e.g., at least 80%, e.g., at least 85%, 90%, or 95%) neurons from the cortex, hippocampus, thalamus, or striatum, or are a mixture of neurons from the cortex and striatum (i.e., cortico-striatal coculture), thalamus and cortex (i.e., thalamo-cortical coculture), or cortex and hippocampus (i.e., cortico-hippocampal coculture).

In preferred embodiments, the cultures are at least 80% neurons, e.g., at least 85%, 90%, or 95% neurons. The cultures may thus contain up to 20% non-neuronal cells, e.g., glial or other cell types. In some embodiments, the cultures contain less than 5% glial cells.

The neurons can be primary neurons, e.g., obtained either from an animal model of such a condition. When primary neurons are used, the inhibitory neurons are preferably medium spiny neurons (i.e., are DARPP32 positive), and the excitatory neurons are CAMKII positive. Alternatively, the neurons can be derived from cells (i.e., from neuronal progenitor cells) from an animal or cellular model or a human, e.g., from an iPS cell made from a human having the condition, or from a stem cell from the human or an animal or cellular model.

Animal models of autism are described in Chomiak and Hu, Neurotoxicol Teratol. 2012 Aug. 28; Nomura and Takumi, Neural Plast. 2012; 2012:589524. doi: 10.1155/2012/589524; Giza et al., J Neurosci. 2010 Nov. 3; 30(44):14805-16; Crawley, Dialogues Clin Neurosci. 2012 September; 14(3):293-305; Bangash et al., Cell. 2011 May 27; 145(5):758-72. Models include knockouts of the FMR1 gene (Iliff et al., “Impaired activity-dependent FMRP translation and enhanced mGluR-dependent LTD in Fragile X premutation mice,” Hum. Mol. Genet. (2012) doi: 10.1093/hmg/dds525; Hagerman et al., Am J Med Genet. 23(1-2):359-74, 1986); the TSC1 gene (Tsai et al., Nature. 2012 Aug. 30; 488(7413):647-51); and ubiquitin protein ligase 3A (Ube3A, Jiang et al., Neuron 21: 799-811, 1988; and Miura et al., Neurobiol Dis. 9: 149-159, 2002). For others see Moy and Nadler, Mol Psychiatry. 13(1):4-26, 2008. Animal models of epilepsy include those described in Guillemain et al., Epileptic Disord. 2012 September; 14(3):217-25; Petrou and Reid, “The GABA_{A}γ2(R43Q) mouse model of human genetic epilepsy.” In: Noebels J L, Avoli M, Rogawski M A, Olsen R W, Delgado-Escueta A V, editors. Jasper's Basic Mechanisms of the Epilepsies [Internet]. 4th edition. Bethesda (Md.): National Center for Biotechnology Information (US); 2012; Steinlein, Expert Rev Neurother. 2010 December; 10(12):1859-67; Sawyer and Escayg, J Clin Neurophysiol. 2010 December; 27(6):445-52; and Coppola and Moshé, Handb Clin Neurol. 2012; 107:63-98. Genetic models for Rett syndrome (methyl-CpG-binding protein-2 (Mecp2)) are described in Amir et al., Nat Genet. 23(2):185-8, 1999). The models described in Mitchell et al., BMC Biology 2011, 9:76, can also be used.

Cellular models include those cells that have been genetically modified to express, overexpress, underexpress, or not express, a wild type or mutant gene associated with the condition, e.g., SHANK3 or islet-brain-2 (IB2) mutants for autism. For example, SHANK3, a gene encoding a postsynaptic scaffolding protein, has been unequivocally implicated in autism and schizophrenia (Prasad et al., Clin Genet 57:103-109, 2000; Precht et al., J Med Genet 35:939-942, 1998; Manning et al., Pediatrics 114, 451-457, 2004; Wilson et al., J Med Genet 40, 575-584, 2003; Jeffries et al., Am J Med Genet A 137:139-147, 2005; Durand et al., Nat Genet 39, 25-27, 2007; Moessner et al., Am J Hum Genet. 81:1289-1297, 2007; Gauthier et al., Am J Med Genet B Neuropsychiatr Genet 150B:421-424, 2009). Loss of function in SHANK3 leads to autistic-like behaviors in mice and humans, e.g., the Phelan-McDermid Syndrome (PMS). Genes encoding other postsynaptic proteins have also been implicated in the pathogenesis of schizophrenia and autism, including the neuroligin-neurexin complex; the PSD95-SAPAP-Shank complex (Tu et al., Neuron 23, 583-592, 1999).

Primary Neuronal Cultures

In cases where brain tissue is available, e.g., from a human cadaver or an experimental animal, e.g., an animal model of a disease associated with neural network dysfunction, primary neurons can be used for the in vitro model. Methods known in the art, e.g., as described herein, are used to dissect and culture striatal and cortical neurons from the animal; see, e.g., Wagenaar et al., BMC Neuroscience 7:11 (2006) (cortical neurons) and Mao and Wang, Methods in Molecular Medicine 79:379-386 (2003) (striatal neurons).

Artificial Neuronal Cultures

In cases where no brain tissue is available, or where cells that are genetically engineered or identical to a given individual are desired, artificial neurons generated by differentiation of stem or progenitor cells can be used.

For example, striatal-like (inhibitory) or cortical-like (excitatory) neuronal cells can be obtained from neural progenitor cells or stem cells (e.g., universal donor hematopoietic stem cells, embryonic stem cells (ES), partially differentiated stem cells, non-pluripotent stem cells, pluripotent stem cells, induced pluripotent stem cells (iPS cells) by methods known in the art; see, e.g., Harwell et al., Neuron. 2012 Mar. 22; 73(6): 1116-26. In some embodiments, the neuronal cells can be derived from induced pluripotent stem cells generated from an epithelial cell from an individual, e.g., an experimental animal (e.g., an animal model of a condition associated with neural network dysfunction, e.g., autism spectrum disorders, schizophrenia, schizoaffective disorder, depression, obsessive-compulsive spectrum disorders, bipolar disorder, or epilepsy) or a human subject (e.g., a subject who has or is suspected of having a condition associated with neural network dysfunction, e.g., autism spectrum disorders, schizophrenia, schizoaffective disorder, depression, obsessive-compulsive spectrum disorders, bipolar disorder, or epilepsy). Methods for making iPS cells from differentiated or adult somatic cells are known in the art, e.g., Yu et al., Science 318 (5858): 1917-1920 (2007); Takahashi et al., Cell 131 (5): 861-872 (2007); Zhou et al., Nature Protocols 7 (12): 2080-2089 (2012). iPS cells have been generated from multiple cells types, including fibroblasts, keratinocytes, peripheral blood, see Zhou et al., Nature Protocols 7 (12): 2080-2089 (2012) and the references cited therein.

Protocols known in the art can be used to differentiate neural progenitor cells or stem cells into striatal-like (inhibitory) or cortical-like (excitatory) neuronal cells. For example, methods for the generation of cortical neurons from pluripotent stem cells are described in Lai et al., Neuron. 2008 Jan. 24; 57(2):232-47; Cruikshank et al., Prog Brain Res. 2005; 149:41-57; Sahara et al., J Neurosci. 2012 Apr. 4; 32(14):4755-61; Song et al., “Coordinated Development of Voltage-gated Na+ and K+ currents Regulates Functional Maturation of Pyramidal-like Neurons Derived from Human iPS Cells.” Stem Cells Dev. 2013; 22:1551-63); Srivastava et al., “Conditional Induction of Mathl Specifies Embryonic Stem Cells to Cerebellar Granule Neuron Lineage and Promotes Differentiation into Mature Granule Neurons,” Stem Cells. 2012 Dec. 7. doi: 10.1002/stem. 1295; Vanderhaeghen, Prog Brain Res. 2012; 201:183-95. Other protocols are described in Walter and Dihné, Front Cell Neurosci. 2012; 6:52; Hick et al., Dis Model Mech. 2012 Nov. 7. [Epub ahead of print] PMID: 23136396; Braun et al., Acta Neurobiol Exp (Wars). 2012; 72(3):219-29; Jiang et al., Protein Cell. 2012 November; 3(11):818-25; Jeon et al., Stem Cells. 2012 September; 30(9):2054-62; Liu and Zhang, Cell Mol Life Sci. 2011 December; 68(24):3995-4008; Lee et al., J Clin Invest. 2011 June; 121(6):2326-35.

In some embodiments, the striatal neurons are derived from cells from an animal or cellular model of, or subject having, the condition, and the cortical neurons are derived from a normal or wild type cell, animal or subject.

The methods can include maintaining the neuronal cultures under conditions suitable for the development of neural networks as are known in the art and described herein.

### Methods of Screening

Included herein are methods for screening test compounds, e.g., polypeptides, polynucleotides, inorganic or organic large or small molecule test compounds, to identify agents useful in the treatment of autism spectrum disorders, or other disorders associated with neural network dysfunction, e.g., schizophrenia and epilepsy.

Assays

The screening methods described herein include the use of techniques that can measure population activity across a whole model network (i.e., “network activity”) substantially simultaneously, as a screening platform to measure synaptic function associated with neuronal network output of live neurons in vitro at scale. A number of methods are known in the art to measure network activity. Suitable methods include the micro-electrode array (MEA) system as well as fluorescence detection, e.g., of intracellular calcium levels.

In a preferred embodiment, a MEA is used. Dissociated cultures of neurons grown on embedded MEAs produce electrical signals over multiple electrodes, and have been used to study network physiology in vitro (Arnold et al., The Journal of physiology. 2005; 564(Pt 1):3-19; Chiappalone et al., Biosens Bioelectron. 2003; 18(5-6):627-34; Hales et al., Frontiers in neural circuits. 2012; 6:29; Haustein et al., J Neurosci Methods. 2008; 174(2):227-36; Jimbo et al., Biophys J. 1999; 76(2):670-8; et al., Conf Proc IEEE Eng Med Biol Soc. 2006; 1:1593-6; Madhavan et al., Conf Proc IEEE Eng Med Biol Soc. 2006; 1:1593-6; Nam and Wheeler, Crit Rev Biomed Eng. 2011; 39(1):45-61; Charvet et al., Conf Proc IEEE Eng Med Biol Soc. 2010; 2010:1804-7; ). These MEA studies described fundamental properties of network activity patterns, plasticity, and learning in vitro, as well as use in pharmacological testing. More important, cortical cells in culture retain many of the properties found in their in vivo context, though significant differences exist (Potter and DeMarese, J Neurosci Methods. 2001; 110(1-2):17-24). Comparing with other techniques studying neuronal output in vitro, MEA has the advantages of being non-invasive, amenable for repeated measures over many days, and easy to operate, having a small footprint, and most importantly reading synaptic function/connection associated with dynamic neural network activities. This system has shown promise for toxin and drug screening (Xiang et al., Biosens Bioelectron. 2007; 22(11):2478-84; Chiappalone et al., Biosens Bioelectron. 2003; 18(5-6):627-34), and safety pharmacology (Meyer et al., Drug Saf. 2004; 27(11):763-72; Easter et al., Drug Discov Today. 2009; 14(17-18):876-84; Stett et al., Anal Bioanal Chem. 2003; 377(3):486-95; J Food Sci. 2008 April; 73(3):E129-36). Although previously used in single well assay formats, the MEA platform is scalable for high throughput use.

Network activity data obtained from an MEA can include parameters from individual electrodes and parameters from population analysis of all electrode in an MEA, e.g., bursts (i.e., activation pattern of neurons where periods of rapid spiking are followed by quiescent, silent, periods; burst can be defined, e.g., by the number of spikes and their rate or frequency), the types of bursts, bursting durations, spike rate within bursts, inter-burst duration, and the number of spikes in each bursts. Synchronizing network behavior can also be quantified using tools in signal processing, such as power spectrum analysis, to recognize the network electric patterns in the frequency domain, e.g., using tools such as MatLab (Mathworks, Natic, Mass.) and Neuroexplorer (Littleton, Mass.). Network oscillation can be quantified, e.g., using autocorrelation or cross-correlation analysis. The distance between different MEA patterns can be calculated to generate signature metrics for screening criteria to identify compounds that have an effect on network activity. For example, the area under the curve (AUC) for specific domain frequencies in the power spectrum analysis can be used to represent the total power of a particular pattern. Alternatively, the area over a specific frequency range can be integrated to calculate the distance between WT and KO MEA spectrum pattern. MEA recordings can also be categorized based on WT and KO training sets, e.g., using pattern recognition software such as Patternz or similar software to extract MEA signatures. Synchronized network patterns generated by autocorrelograms and crosscorrelogram among electrodes within an MEA can be used to represent the total power at slow or fast network oscillations. Patterns of principle component analysis of network activities can be used to represent the network dynamics and stabilities. These and other methods of analysis are described in the literature, see, e.g., Kalitzin et al., Biol Cybern. 1997 January; 76(1):73-83; Christopoulos et al., J Neural Eng. 2012 August; 9(4):046008; Borisyuk et al., Biol Cybern. 1985; 52(5):301-6; Esposti et al., IEEE Trans Neural Syst Rehabil Eng. 2009 August; 17(4):364-9; Stephens et al., Exp Neurol. 2012 March; 234(1):20-30; Frohlich and McCormick. Neuron 2010 Jul. 15 67(1) 129-43; Tahvildari et al, J Neurosci. 2012 Aug. 29; 32(35):12165-79.

In some embodiments, network activity data is obtained from a single culture under multiple conditions, e.g., at different times in culture, or in the presence or absence of one or more agents, e.g., test compounds and/or positive and negative controls. Agents that are known to have a given effect can be used as positive or negative controls. For example, agents that are known to stimulate a certain response can be used to induce a given level of activity (e.g., an excited signature); such agents can include pharmacological agents such as picrotoxin (PTX), Semicarbazide (a Glutamic Acid Decarboxylase inhibitor) or electrical stimuli. In some embodiments, a channelrhodopsin (CR2)-based approach is used to uniformly activate the culture by light stimulation (480 nM). Thus in some embodiments, CR2 cDNA is introduced to the culture before plating (e.g., using electroporation or lentiviral delivery). CR2 can be driven by a specific promoter to produce expression only in specific neurons; see, e.g., Schonig et al., BMC Biol. 2012 Sep. 3; 10:77; Magen and Chesselet, Prog Brain Res. 2010; 184:53-87. For example, CR2 driven by CAMKII is only expressed in excitatory neurons; the CAMKII promoter is known in the art and is commercially available, e.g., in the a-CaMKII promoter-based vectors FCK(0.4)GW and FCK(1.3)GW (Stratagene). CR2 proteins are photosensitive, so exposing the neuronal culture to light results in depolarization of CR2 in neurons that express CR2, therefore activating these neurons that contain CR2. Thus, as CR2 has the ability to stimulate specific circuits via specific promoters, specific neurons in the network can be activated using this strategy. Agents that are known to quench network activity can also be used, e.g., at the end of each experiment, to obtain activity data under quiescent conditions (e.g., a quiescent signature); such agents include the sodium channel blockers such as tetrodotoxin (TTX), NMDA receptor inhibitors, e.g., MK801, or AMPA receptor inhibitors, e.g., CNQX. A normal level of network activity obtained in the absence of either stimuli or quenching agents can be referred to as a baseline signature.

Agents that reduce network activity in the stimulated state (e.g., reduce network activity in the excited signature, such that it appears more like the baseline or quenched signature) are considered hits.

Test Compounds

The methods described herein can include evaluating the models under one or more conditions, e.g., contacting the models with one or more test compounds, at one or more doses. The test conditions can include variations in the components of the media. The test compounds can be, e.g., any organic or inorganic compounds, including nucleic acids (e.g., to result in overexpression or knockout of a gene, e.g., a transgene, siRNA, shRNA, antisense, etc), proteins, peptides, pathogens, or small molecules.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in some embodiments a general library of small molecules is screened, e.g., using the methods described herein.

Combinations of test compounds can also be tested, thus, the methods can include contacting the model with two or more test compounds, either sequentially or substantially simultaneously. These methods can be used, e.g., to detect drug interactions or synergistic effects of test compounds. In addition, two or more different doses can be used and a dose-response curve optionally prepared, e.g., to estimate an effective dose for administration to a subject.

In some embodiments, the test compound is a drug that is a known or suspected treatment for a condition described herein. For example, anti-psychotics, anti-depressants, anti-seizure medications or others as are known in the art can be evaluated using the present methods, e.g., antipsychotic medications, e.g., aripiprazole, chlorpromazine, clozapine, fluphenazine, haloperidol, iloperidone, loxapine, molindone, olanzapine, paliperidone, perphenazine, pimozide, quetiapine, risperidone, thioridazine, thiothixene, trifluoperazine, ziprasidone; antidepressant medications (also used for anxiety disorders), e.g., amitriptyline (tricyclic), amoxapine, bupropion, citalopram (ssri), clomipramine (tricyclic), desipramine (tricyclic), desvenlafaxine (snri), doxepin (tricyclic), duloxetine (snri), escitalopram (ssri), fluoxetine (ssri), fluoxetine (ssri), fluvoxamine (ssri), imipramine (tricyclic), imipramine pamoate (tricyclic), isocarboxazid (maoi), maprotiline (tricyclic), mirtazapine, nortriptyline (tricyclic), paroxetine (ssri), paroxetine mesylate (ssri), phenelzine (maoi), protriptyline (tricyclic), selegiline, sertraline (ssri), tranylcypromine (maoi), trazodone, trimipramine (tricyclic), venlafaxine (snri); mood stabilizing and anticonvulsant medications, e.g., carbamazepine, divalproex sodium (valproic acid), gabapentin, lamotrigine, lithium carbonate, lithium citrate, oxcarbazepine, topiramate; anti-anxiety medications (all of these are benzodiazepines, except buspirone), e.g., alprazolam, buspirone, chlordiazepoxide, clonazepam, clorazepate, diazepam, lorazepam, oxazepam; ADHD medications, (all of these adhd medications are stimulants, except atomoxetine and guanfacine), e.g., amphetamine, amphetamine, atomoxetine, dexmethylphenidate, dexmethylphenidate, dextroamphetamine, guanfacine, lisdexamfetamine dimesylate, methamphetamine, and methylphenidate; and anti-seizure medications, e.g., carbamazepine, ethosuximide, felbamate, tiagabine, levetiracetam, lamotrigine, pregabalin, gabapentin, phenytoin, topiramate, and oxcarbazepine; and baclofen. Combinations thereof can also be tested to identify any with synergistic effects.

In general, a test compound is applied to an in vitro model of neural network dysfunction as described herein, and one or more effects of the test compound is evaluated, i.e., an effect on an aspect of electrical activity in the network. Data regarding the effect of a test compound can be obtained both in the presence of a network stimulus and the absence of a stimulus, as described above.

A test compound that has been screened by a method described herein and determined to ameliorate network dysfunction, e.g., by returning a network exhibiting high levels of activity (e.g., an excited signature) to a more normal or below-normal level of activity (e.g., a baseline or quiescent signature) can be considered a candidate compound. A candidate compound that has been screened, e.g., in an in vivo model of a disorder, e.g., associated with neural network dysfunction, e.g., autism spectrum disorders, schizophrenia, schizoaffective disorder, depression, obsessive-compulsive spectrum disorders, bipolar disorder, or epilepsy, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that positively affect electrical activity in the in vitro model described herein) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with neural network dysfunction, e.g., autism, schizophrenia or epilepsy. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with neural network dysfunction, e.g., autism, schizophrenia or epilepsy, as described herein. The animal can be monitored for a change in the disorder, e.g., for an improvement in a parameter of the disorder, e.g., a parameter related to clinical outcome. In some embodiments, the animal is a model for autism and the parameter is motor impairment, tremors, hypoactivity, aberrant hindlimb clasping, and/or respiratory symptoms, and an improvement would be a return to normal. In some embodiments, the subject is a human, e.g., a human with autism, and the parameter is abnormal social interaction and communication, aberrant repetitive behavior, unusual motoric responses, such as tic-like stereotypies or self-injury, impaired motor function, abnormal responses to sensory stimuli, language deficits, rigid adherence to routines and restricted interests, obsessional preoccupations (e.g., with activities or things), seizures, anxiety, and/or sleep disorders. See, e.g., Moy et al., American Journal of Medical Genetics Part C (Semin. Med. Genet.) 142C:40-51 (2006).

### Methods of Diagnosis

Included herein are methods for diagnosing a disorder associated with neural network dysfunction, e.g., autism spectrum disorders, schizophrenia, schizoaffective disorder, depression, obsessive-compulsive spectrum disorders, bipolar disorder, or epilepsy. The methods include obtaining a sample comprising cells from a subject, using the cells to generate an in vitro model of neural network activity as described herein, evaluating electrical activity in the model, and comparing the activity in the model with activity in one or more references, e.g., a control reference that represents a normal level of activity, e.g., a level in an in vitro model from an unaffected subject, and/or a disease reference that represents a level of activity associated with neural network dysfunction, e.g., a level in a subject having autism spectrum disorders, schizophrenia, schizoaffective disorder, depression, obsessive-compulsive spectrum disorders, bipolar disorder, or epilepsy. The level of activity in a model can be evaluated using methods known in the art, as described herein.

In some embodiments, the level of activity in the model is comparable to the level of activity in the disease reference, and the subject has one or more symptoms associated with autism spectrum disorders, schizophrenia, schizoaffective disorder, depression, obsessive-compulsive spectrum disorders, bipolar disorder, or epilepsy, then the subject is diagnosed with autism spectrum disorders, schizophrenia, schizoaffective disorder, depression, obsessive-compulsive spectrum disorders, bipolar disorder, or epilepsy. In some embodiments, the subject has no overt signs or symptoms of autism spectrum disorders, schizophrenia, schizoaffective disorder, depression, obsessive-compulsive spectrum disorders, bipolar disorder, or epilepsy, but the level of activity in the subject-derived model is comparable to the level of activity in the disease reference, then the subject has an increased risk of developing autism spectrum disorders, schizophrenia, schizoaffective disorder, depression, obsessive-compulsive spectrum disorders, bipolar disorder, or epilepsy. In some embodiments, the sample includes epithelial cells or stem cells from the subject, and methods known in the art and described herein are used to generate an in vitro model. In some embodiments, once it has been determined that a person has autism spectrum disorders, schizophrenia, schizoaffective disorder, depression, obsessive-compulsive spectrum disorders, bipolar disorder, or epilepsy, or has an increased risk of developing autism spectrum disorders, schizophrenia, schizoaffective disorder, depression, obsessive-compulsive spectrum disorders, bipolar disorder, or epilepsy, then a treatment, e.g., as known in the art or as described herein, can be administered.

The predetermined level of activity can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g., autism spectrum disorders, schizophrenia, schizoaffective disorder, depression, obsessive-compulsive spectrum disorders, bipolar disorder, or epilepsy).

A disease reference subject is one who has (or has an increased risk of developing) autism spectrum disorders, schizophrenia, schizoaffective disorder, depression, obsessive-compulsive spectrum disorders, bipolar disorder, or epilepsy. An increased risk is defined as a risk above the risk of subjects in the general population.

The predetermined value can depend upon the particular population of subjects (e.g., human subjects) selected. For example, an apparently healthy population will have a different ‘normal’ range of levels of activity than will a population of subjects which have, or are likely to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art. In characterizing likelihood, or risk, numerous predetermined values can be established.

### Methods of Treatment

The methods described herein include methods for the treatment of subjects diagnosed with, or at risk of developing, a disorder associated with neural network dysfunction, e.g., autism spectrum disorders, schizophrenia, schizoaffective disorder, depression, obsessive-compulsive spectrum disorders, bipolar disorder, or epilepsy, by a method described herein. In some embodiments, the disorder is autism; in some embodiments, the disorder is Phelan-McDermid Syndrome (PMS). Generally, the methods include administering a treatment as known in the art or described herein, to a subject who is in need of, or who has been determined to be in need of, such treatment.

Conventional treatments include behavioral training and management (e.g., Applied Behavioral Analysis (ABA), Treatment and Education of Autistic and Related Communication Handicapped Children (TEACCH), and/or sensory integration); speech, occupational, and/or physical therapy; pharmaceuticals/drug treatment (e.g., selective serotonin reuptake inhibitors (SSRIs); antipsychotic agents such as haloperidol (Haldol), risperidone (Risperdal), and thioridazine; Clonidine (Catapres); guanfacine hydrochloride (Tenex); Lithium (Eskalith, Eskalith-CR, Lithobid); and anticonvulsants, such as carbamazepine (Carbatrol, Epitol, Tegretol) and valproic acid (Depakene)).

In some embodiments, the methods include obtaining a sample comprising cells from a subject, using the cells to generate an in vitro model of neural network activity as described herein, evaluating electrical activity in the model, and comparing the activity in the model with activity in the model in the presence of one or more potential treatments. A treatment that is effective in reducing aberrant activity in the subject's model can then be selected and optionally administered to the subject. The level of activity in a model can be evaluated using methods known in the art, as described herein.

### Examples

The invention is further described in the following examples, which do not limit the scope of the invention described in the claims.

Materials and Methods

The following materials and methods were used in the Examples set forth herein, unless otherwise indicated.

Cell Culture

Primary dissociated cortical neurons were prepared from postnatal day 0 wild type and Shank3 KO mice (Peca et al., 2011) from littermates using standard protocols as described before (Ramamoorthi et al., 2011). For micro-electrode array recordings, dissociated neurons were plated onto substrate embedded electrodes pre-coated with polyethyleneimine (PEI) (Sigma-Aldrich, 0.1%) plus laminin (Life Technologies, 20 μg/ml) at a density of 3×10³ cells/mm². For biochemical experiments, 3×10⁵ cells were plated onto 1 well of the 24-well plate pre-coated with 20 μg/ml Poly-D-lysine (PDL, Sigma-Aldrich) and 4 μg/ml laminin (Life Technologies). The cultures were treated with AraC (Sigma-Aldrich, 1 μg/ml) at day 5 in vitro, and maintained up to 21 days after plating.

MEA Recordings

The electrical activity of cultured neurons was recorded using MEA2100-Systems (Multichannel Systems) with integrated amplifier. Each MEA dish (6-well MEA 200/30 iR-Ti-rcr, Multichannel Systems) contains six wells, with 9 microelectrodes (TiN, 30 μm diameter) arranged over a 3×3 square grid at 200 μm inter-electrode distance in each MEA well (FIG. 1C). Recordings started 10 minutes after the MEA plates were placed to the headstage, which was set to 35° C., and the culture was supplied with continuous perfusion of 5% carbon dioxide balanced air (Airgas) throughout recording. For all time points earlier than 21 days in vitro (DIV21), MEA recordings lasted 15 minutes. At DIV21, MEA recordings lasted 30 minutes. All MEA recordings were performed in culture medium without perfusion. The electric signals were collected at 10 kHz with MCRack (Multichannel Systems; Version 4.4.2) and analyzed offline.

MEA Data Analysis

Spike Detection

The software MCRack was used for spike detection. Raw MEA data was first high-pass filtered at 200 Hz to remove the low-frequency local field potentials. Spikes were detected using a threshold-based detector set to as upward or downward excursion beyond 5.5x the standard deviations (SD, calculated from 500 ms of filtered data that did not contain spike activity) above the peak-peak noise level (Wagenaar et al., 2006). The average detection threshold is around 16 μV. Spike sorting was not performed, thus multiunit activity may contribute to the spikes detected from one electrode. Timestamps of spikes were stored and used for further analysis.

Array-Wide Spike Detection Rate (ASDR) Plot Analysis

ASDR plot was generated using the method modified from Wagenaar et al. (2008) by summing the number of spikes detected per 200 ms from all electrodes in each well and plotted in time series. Clampfit 10.2 (Molecular Devices) was used for ASDR waveform analysis. 5% of individual ASDR's maximum peak value was calculated and set as the detecting threshold for identifying ASDR spike. As shown in FIG. 3, any single ASDR wave above the detecting threshold was defined as ASDR spike, and the duration between two sequential ASDR spikes was defined as inter-spike interval (ISI). ASDR spikes with ISIs below 10 seconds were grouped together and defined as network active period (NAP). The duration between two sequential NAP states was defined as network inactive period (NIP).

Pharmacological Experiments

All drugs were dissolved in DMSO to make stock solutions before applying to neurons in culture media for pharmacological studies. Picrotoxin (PTX, stock solution of 100 mM) for blocking GABA_{A} receptors (Tocris), Clonazepam (stock solution of 30 mM) as GABA_{A} receptor positive modulator (Sigma), NBQX (stock solution of 10 mM) for blocking AMPA receptor (Abcam), and CX546 (stock solution of 100 mM) as AMPA receptor potentiator (Tocris) were used in doses as described herein. The maximum concentration of DMSO in the culture media with drug application was 0.08% volume.

Immunocytochemistry

Neurons were fixed by incubation with 4% paraformaldehyde and 4% sucrose in phosphate-buffered saline (PBS) at room temperature for 5 minutes, and washed three times in PBS. Fixed neurons were permeabilized with 0.1% Triton X-100/PBS for 5 minutes, washed three times with PBS, and incubated in 3% bovine serum albumin plus 10% normal goat serum in PBS for 1 hour to block nonspecific binding. Cells were then incubated overnight with the following primary antibodies at 4° C.: mouse anti-NeuN (Millipore, 1:1000) and rabbit anti-GABA (Sigma, 1:1000). After washing with PBS three times, cells were incubated with indocarbocyanine (Cy3)-conjugated secondary antibodies (Jackson ImmunoResearch Laboratories, 1:1000) or with Alexa 488-conjugated secondary antibodies (Jackson ImmunoResearch Laboratories, 1:1000).

Western Blot

Whole cell lysates were prepared for western blot as described previously (Shcheglovitov et al., 2013). Equal amounts (10 μg) of samples were loaded onto 4-12% SDS-PAGE gel and transferred to a nitrocellulose membrane by electroblotting. The membranes were blocked with Odyssey Blocking Buffer for 1 hour at room temperature. After blocking, membranes were incubated overnight in the primary antibody dilution at 4° C. (anti-Shank3, Santa Cruz, 1:500; anti-P3-Actin, Sigma, 1:50,000). The blots were washed four times with TBST and incubated with the IRDye (800 CW or 700 CW)-labeled secondary antibody (1:5000) for 1 hour at room temperature. The membrane was washed four times with TBST and then scanned with the LI-COR Odyssey Infrared Imaging System (LI_COR Biotechnology).

GCaMP Imaging

AAV2/8-CaMKII-GCaMP6F virus (the titer of virus is 1×10¹² virus particles per milliliter, 0.2 μl per well) was added to neuronal culture at DIV11 and GCaMP imaging was performed 10 days after virus infection. Fluorescent signals were imaged by a confocal microscope (Fluoview FV 1000; Olympus) with a 30 mW multiline argon laser at 5-10% laser power. The laser with a wavelength of 488 nm was used for excitation, and fluorescence was recorded through a band-pass filter (505-525 nm). The images were acquired using 10x objective lens with 2 Hz scanning speed. XYT image galleries were collected and average fluorescence intensity in the soma of was measured for the quantification by custom written Matlab (MathWorks) program. Each GCaMP trace represents the fluorescence signals detected from the soma of one neuron. We report time series as ΔF/F=(F−F0)/F0, where F is the raw fluorescence signal, F0 is the basal fluorescence signal, 15 percentile of fluorescence signal.

### Example 1. MEA Recordings Capture Spontaneous Cortical Network Activities In Vitro

To study the firing activities of in vitro network, we cultured dissociated wild type (WT) cortical neurons derived from postnatal day 0 mice up to 21 days in vitro (DIV) at high density (3×10³ cells/mm²) on substrate-embedded electrodes and performed MEA recordings at multiple developmental ages. As shown in FIG. 1A, electric signals generated from neurons were captured by micro-electrodes. Individual action potentials (spikes) were identified by threshold-based spike detection. The spikes were time-stamped, used for raster plots and subsequent data analysis. As shown in the representative raster plots from one WT cortical culture over development (FIG. 1B), there are several common electric features observed from such in vitro neural networks. First, spikes from multiple electrodes at different locations within the same network displayed synchronized firings at and after DIV7. Second, as the cultures developed, the mean spike rate significantly increased from DIV7 to DIV21 (FIG. 6), indicating that the network output progressively increased during the development in vitro, reaching the plateau at the end of three weeks. Third, at and after DIV18, these in vitro networks displayed a distinct firing pattern with long active firing period followed by prolonged inactive period that lasted for tens of seconds (FIG. 1C).

We did not go beyond three weeks for recording to ensure the high quality of the cultures at such high plating density. Compared with earlier reports of hippocampal networks where spontaneous firing frequency plateaued at DIV11-13 (Bateup et al., 2013), the cortical network spontaneous firing activities plateaued at a much later time, towards DIV21. Such differences may reflect the source of the neurons (E18 vs P0), culture conditions, and intrinsic differences between cortical network and hippocampal network in vitro.

Since MEA does not pre-select the neurons to record, spiking activities recorded by MEA from cortical cultures might come from either excitatory or inhibitory neurons. In order to understand what type of neuronal output, excitatory or inhibitory, dominate the observed synchronized cortical networks detected by MEA, we infected cortical cultures with AAV2/8-CaMKII-GCaMP6F to drive GCaMP6F, a genetically encoded calcium indicator, expression selectively in excitatory neurons and performed simultaneous GCaMP imaging and MEA recordings from the same culture (FIG. 1D). The firing activities recorded by MEA, as shown in raster plot, corresponded well with the Ca²⁺ oscillation observed by GCaMP imaging (FIG. 1E), indicating that the observed MEA recordings from cortical cultures came from the firings of excitatory neurons. As a result, spiking activities recorded by MEA reflect the overall excitatory output of the network.

### Example 2. Shank3 KO Cortical Neurons Produce Reduced-Firing in Spontaneous and PTX-Treated Networks

To investigate whether the deletion of Shank3 gene affects spontaneous firings of cortical neurons in culture, we then performed MEA recordings from Shank3 KO cultures with age in vitro. Similar to WT neurons, Shank3 KO cortical neurons in culture displayed synchronized firings at and after DIV7 (FIG. 1C). Simultaneous CaMKII-GCaMP6F imaging and MEA recordings revealed MEA recordings from Shank3 KO cultures also reflect the excitatory output of the network (FIG. 1F). While no difference in spike rate was observed prior to and at DIV14, Shank3 KO cultures showed significantly reduced spike rate at DIV18 and DIV21 compared to the WT cultures (FIGS. 2A and 2B), and also reduction in bursting activities (FIG. 7). It is interesting to note that the difference in the spiking rate and burst properties between WT and Shank3 KO only developed later in vitro (after DIV14). This may reflect the fact that the expression of Shank3 protein in cortical cultures is development dependent and increased 2 folds between DIV7 and DIV14 (FIG. 9).

It has been shown that the number of total neurons in the network influence the firing activity of cortical cultures (Wagenaar et al., 2006). To evaluate whether the observed reduced-firing network from Shank3 KO group was due to different amount of neurons in the network, we performed immunocytochemistry using the same high density cultures and found no difference in the number of total neurons (as stained with NeuN) or the number of GABAergic neurons (as stained with GABA) in Shank3 KO cultures compared to WT cultures at DIV21 (FIG. 10).

Since reduced excitatory synaptic transmission in the absence of Shank3 protein has been shown previously in mouse brain slices and induced human neurons (Peca et al., 2011; Shcheglovitov et al., 2013), we then treated our Shank3 KO cultures with AMPA receptor positive modulator CX546 and found CX546 (30 μM) rescued the reduced-firing in Shank3 KO networks (FIG. 2C), suggesting that reduced excitation underlies the hypofiring Shank3 KO network. To further exam the firing activities of WT and Shank3 KO cultures with only excitatory input and output, we then treated both WT and Shank3 KO networks with GABA_{A} receptor antagonist picrotoxin (PTX) at 50 μM to fully block the inhibitory synaptic transmission. As shown in FIG. 2E, treating the networks with PTX significantly increased the mean spike rate in both WT and Shank3 KO cultures. Similar to what we observed from the spontaneous networks, PTX-treated Shank3 KO networks were also with significantly reduced firings compared to PTX-treated WT networks (FIGS. 2D, 2E, and 2F). CX546 (30 μM) again effectively rescued the reduced-firing in the PTX-treated Shank3 networks (FIG. 2F). Together, these data suggest that reduced excitatory synaptic transmission is the predominant cause of reduced-firing in cortical cultures in the absence of Shank3 protein.

### Example 3. Analyze Global Network Firing Patterns with ASDR Plot

As mentioned in FIG. 1B, WT cortical cultures started to display a distinct spontaneous network firing pattern with long active period followed by prolonged quiet period (with no firing activities) at or after DIV18. Although similar global network firing pattern has been documented in the study of cortical cultures derived from embryonic rat E18 neurons (Wagenaar et al., 2006), no quantification has been reported to analyze such bi-stable states. We found that an ASDR plot, generated by summing the number of spikes detected per unit time over all electrodes from the same culture (Wagenaar et al., 2006), robustly captured the network activities across the entire network seen in the raster plot (FIG. 3A). We then define network active period (NAP) and network inactive period (NIP) to capture the network oscillation between active and quiet states (described in more detail in methods), and used the duration of NAP and NIP for analyzing global network firing-patterns.

### Example 4. Cultured Shank3 KO Cortical Neurons Display Altered Global Network Firing-Pattern

Using the duration of NAP and NIP, we compared the global network firing-pattern between WT and Shank3 KO cortical cultures at DIV21. First of all, at DIV21, fewer wells of Shank3 KO cultures (38 out of 50 cultured networks, 73%) developed NAP/NIP alternation compared to the WT cultures (49 out of 50 cultured networks, 98%). For those Shank3 KO cultures without NAP/NIP alternations (27%), no NIPs longer than 10 seconds could not be detected from the ASDR plot (FIG. 8). Second, we found that the average duration for both NAP and NIP from Shank3 KO cultures was significantly shorter compared to that from WT cultures (FIGS. 3B and 3C). The cumulative distribution curve for the duration of NAP and NIP clearly separated between WT and Shank3 KO groups (FIG. 3C).

### Example 5. The Duration of NIP is Sensitive to the Change in the Excitatory/Inhibitory Synaptic Transmission

To understand how excitatory and inhibitory synaptic transmission contribute to the network firing pattern, we next evaluated the change of NAP and NIP upon reciprocal regulating the synaptic strength with NBQX (AMPA receptor antagonist to reduce excitatory synaptic transmission), CX546 (AMPA receptor positive modulator to enhance excitatory synaptic transmission), picrotoxin (PTX, GABA_{A} receptor antagonist to reduce inhibitory synaptic transmission) and clonazepam (CLP, GABA_{A} receptor positive modulator to enhance inhibitory synaptic transmission). Multiple sub-saturating doses for each compound were chosen based on previous reports (Mienville et al., 1989; Nagarajan et al., 2001; Lu et al., 2006; Han et al., 2014; Han et al., 2012; Huntsman et al., 2006), and their effects were evaluated using MEA recordings. As shown in FIG. 4, the duration for NIP is very sensitive to both excitatory and inhibitory strength in the network. Enhancing inhibition by CLP dose dependently elongated the NIP duration, reaching 42±14% longer NIP at 100 nM (FIG. 4A). In contrast, PTX treatment reduced the NIP duration to 51±6% (at 0.2 μM) and 44±6% (at 0.5 μM) compared to basal (FIG. 4B). In comparison, changing excitatory synaptic strength showed opposite effect: CX546 significantly decreased the NIP duration to 58±11% at 30 μM (FIG. 4C), whereas NBQX significantly enhanced the NIP duration by 560±85% at 1 μM (FIG. 4D). Taken together, these data indicate that NIP duration is very sensitive to the relative strength of excitatory and inhibitory input to the network; either reducing inhibition or enhancing excitation reduced the duration of NIP. Conversely, either enhancing inhibition or reducing excitation increased the NIP duration. These results demonstrate that the NIP duration reflects the balance between excitatory and inhibition in the network, and that small changes in excitatory or inhibitory synaptic strength will induce the change in NIP duration.

Interestingly, the duration of NAP was not as sensitive as the duration of NIP to the change in excitation or inhibition. For example, the length of NAP remained unchanged upon PTX treatment up to 0.5 μM or CX546 treatment up to 30 μM, suggesting NAP is not as sensitive as NIP to synaptic perturbations (FIGS. 11A-11D).

### Example 6. Altered Shank3 KO Network Firing Pattern can be Rescued by Changing Inhibitory but not Excitatory Synaptic Transmission

To test whether increasing excitatory synaptic transmission could rescue the defect in the network patterns as it did in reduced-firing activity (FIG. 2), we treated Shank3 KO cultures with CX546 and discovered that increasing excitatory transmission did not significantly change the duration of NAP (FIG. 11E) and NIP (FIG. 5A) in Shank3KO networks, suggesting that the deficit in network firing pattern with Shank3 deletion was not under the influence of excitatory synaptic strength. It has been shown before that the development of prolonged network quiet period in cultured networks was linked to increasing amount of inhibitory input in the culture (Chen et al., 2010). Recently, reduced parvalbumin (PV)+ puncta has been reported in the insular cortex for Shank3 KO mice (Gogolla et al., 2014). We then treated Shank3 KO cultures with GABA_{A} positive modulator Clonazepam (CLP) to enhance inhibitory circuit function, and found CLP dose dependently increased NIP duration, and normalized the shortened NIP in the Shank3 KO network to the level of WT at 100 nM (FIG. 5B). These data suggest that weakened inhibitory synaptic transmission in Shank3 KO neurons contribute to the observed global network pattern deficit for NIP duration.

In fact, from evaluating the response of WT networks to pharmacological manipulation of excitatory/inhibitory synaptic transmission (FIG. 4), we already learned that manipulating WT networks by enhancing excitation or reducing inhibition produced shortened NIP duration and faster network oscillation (FIGS. 4B and 4C), which is similar to the network defect identified in Shank3 KO neurons (FIG. 3). Coupled with the result that CLP effectively rescued the NIP durations in the Shank 3 KO networks, the global network oscillation defect in the Shank3 KO network can, at least partially, be attributed to decreased GABAergic function in the cultured micro-circuitry. The data demonstrated for the first time that MEA recording revealed reductions in both excitation and inhibition in the Shank3 KO autism model in vitro.

### REFERENCES

Aladjalova N A. Infra-slow rhythmic oscillations of the steady potential of the cerebral cortex. Nature 1957; 179(4567): 957-959.
Baird G, Simonoff E, Pickles A, Chandler S, Loucas T, Meldrum D et al. Prevalence of disorders of the autism spectrum in a population cohort of children in South Thames: the Special Needs and Autism Project (SNAP). Lancet 2006; 368(9531): 210-215.
Baron M K, Boeckers T M, Vaida B, Faham S, Gingery M, Sawaya M R et al. An architectural framework that may lie at the core of the postsynaptic density. Science 2006; 311(5760): 531-535.
Bateup H S, Johnson C A, Denefrio C L, Saulnier J L, Kornacker K, Sabatini B L. Excitatory/inhibitory synaptic imbalance leads to hippocampal hyperexcitability in mouse models of tuberous sclerosis. Neuron 2013; 78(3): 510-522.
Boeckers T M, Bockmann J, Kreutz M R, Gundelfinger E D. ProSAP/Shank proteins—a family of higher order organizing molecules of the postsynaptic density with an emerging role in human neurological disease. Journal ofneurochemistry 2002; 81(5): 903-910.
Cao Z, Hulsizer S, Tassone F, Tang H T, Hagerman R J, Rogawski M A et al. Clustered burst firing in FMR1 premutation hippocampal neurons: amelioration with allopregnanolone. Human molecular genetics 2012; 21(13): 2923-2935.
Chao H T, Chen H, Samaco R C, Xue M, Chahrour M, Yoo J et al. Dysfunction in GABA signalling mediates autism-like stereotypies and Rett syndrome phenotypes. Nature 2010; 468(7321): 263-269.
Chen X, Dzakpasu R. Observed network dynamics from altering the balance between excitatory and inhibitory neurons in cultured networks. Physical review E, Statistical, nonlinear, and soft matter physics 2010; 82(3 Pt 1): 031907.
Chiappalone M, Bove M, Vato A, Tedesco M, Martinoia S. Dissociated cortical networks show spontaneously correlated activity patterns during in vitro development. Brain Research 2006; 1093(1): 41-53.
Compte A, Sanchez-Vives M V, McCormick D A, Wang X J. Cellular and network mechanisms of slow oscillatory activity (<1 Hz) and wave propagations in a cortical network model. Journal of Neurophysiology 2003; 89(5): 2707-2725.
Crunelli V, Hughes S W. The slow (<1 Hz) rhythm of non-REM sleep: a dialogue between three cardinal oscillators. Nature Neuroscience 2010; 13(1): 9-17.
Durand C M, Betancur C, Boeckers T M, Bockmann J, Chaste P, Fauchereau F et al. Mutations in the gene encoding the synaptic scaffolding protein SHANK3 are associated with autism spectrum disorders. Nature Genetics 2007; 39(1): 25-27.
Fromer M, Pocklington A J, Kavanagh D H, Williams H J, Dwyer S, Gormley P et al. De novo mutations in schizophrenia implicate synaptic networks. Nature 2014; 506(7487): 179-184.
Gauthier J, Spiegelman D, Piton A, Lafreniere R G, Laurent S, St-Onge J et al. Novel de novo SHANK3 mutation in autistic patients. American journal of medical genetics Part B, Neuropsychiatric Genetics: the official publication of the International Society of Psychiatric Genetics 2009; 150B(3): 421-424.
Gogolla N, Leblanc J J, Quast K B, Sudhof T C, Fagiolini M, Hensch T K. Common circuit defect of excitatory-inhibitory balance in mouse models of autism. Journal of Neurodevelopmental Disorders 2009; 1(2): 172-181.
Gogolla N, Takesian A E, Feng G, Fagiolini M, Hensch T K. Sensory Integration in Mouse Insular Cortex Reflects GABA Circuit Maturation. Neuron 2014; 83(4): 894-905.
Gray C M, Konig P, Engel A K, Singer W. Oscillatory responses in cat visual cortex exhibit inter-columnar synchronization which reflects global stimulus properties. Nature 1989; 338(6213): 334-337.
Hales C M, Zeller-Townson R, Newman J P, Shoemaker J T, Killian N J, Potter S M. Stimulus-evoked high frequency oscillations are present in neuronal networks on microelectrode arrays. Frontiers in neural circuits 2012; 6: 29.
Han S, Tai C, Westenbroek R E, Yu F H, Cheah C S, Potter G B et al. Autistic-like behaviour in Scnla+/−mice and rescue by enhanced GABA-mediated neurotransmission. Nature 2012; 489(7416): 385-390.
Han S, Tai C, Jones C J, Scheuer T, Catterall W A. Enhancement of inhibitory neurotransmission by GABA_{A} receptors having alpha2,3-subunits ameliorates behavioral deficits in a mouse model of autism. Neuron 2014; 81(6): 1282-1289.
Hayashi M K, Tang C, Verpelli C, Narayanan R, Steams M H, Xu R M et al. The postsynaptic density proteins Homer and Shank form a polymeric network structure. Cell 2009; 137(1): 159-171.
Hempel C M, Sivula M, Levenson J M, Rose D M, Li B, Sirianni A C et al. A system for performing high throughput assays of synaptic function. PloS ONE 2011; 6(10): e25999.
Holcman D, Tsodyks M. The emergence of Up and Down states in cortical networks. PLoS computational biology 2006; 2(3): e23.
Huntsman M M, Huguenard J R. Fast IPSCs in rat thalamic reticular nucleus require the GABA_{A} receptor beta1 subunit. Journal of Physiology 2006; 572(Pt 2): 459-475.
Iossifov I, O'Roak B J, Sanders S J, Ronemus M, Krumm N, Levy D et al. The contribution of de novo coding mutations to autism spectrum disorder. Nature 2014; 515(7526): 216-221.
Jimbo Y, Tateno T, Robinson H P. Simultaneous induction of pathway-specific potentiation and depression in networks of cortical neurons. Biophysical Journal 1999; 76(2): 670-678.
Lu C, Fu Z, Karavanov I, Yasuda R P, Wolfe B B, Buonanno A et al. NMDA receptor subtypes at autaptic synapses of cerebellar granule neurons. Journal of neurophysiology 2006; 96(5): 2282-2294.
Maeda E, Robinson H P, Kawana A. The mechanisms of generation and propagation of synchronized bursting in developing networks of cortical neurons. The Journal of Neuroscience: the official journal of the Society for Neuroscience 1995; 15(10): 6834-6845.
Mienville J M, Vicini S. Pregnenolone sulfate antagonizes GABA_{A} receptor-mediated currents via a reduction of channel opening frequency. Brain Research 1989; 489(1): 190-194.
Mok S Y, Nadasdy Z, Lim Y M, Goh S Y. Ultra-slow oscillations in cortical networks in vitro. Neuroscience 2012; 206: 17-24.
Nagarajan N, Quast C, Boxall A R, Shahid M, Rosenmund C. Mechanism and impact of allosteric AMPA receptor modulation by the ampakine CX546. Neuropharmacology 2001; 41(6): 650-663.
Peca J, Feliciano C, Ting J T, Wang W, Wells M F, Venkatraman T N et al. Shank3 mutant mice display autistic-like behaviours and striatal dysfunction. Nature 2011; 472(7344): 437-442.
Precht K S, Lese C M, Spiro R P, Huttenlocher P R, Johnston K M, Baker J C et al. Two 22q telomere deletions serendipitously detected by FISH. Journal of Medical Genetics 1998; 35(11): 939-942.
Ramamoorthi K, Fropf R, Belfort G M, Fitzmaurice H L, McKinney R M, Neve R L et al. Npas4 regulates a transcriptional program in CA3 required for contextual memory formation. Science 2011; 334(6063): 1669-1675.
Ripke et al. Biological insights from 108 schizophrenia-associated genetic loci. Nature 2014; 511(7510): 421-427.
Rubenstein J L, Merzenich M M. Model of autism: increased ratio of excitation/inhibition in key neural systems. Genes, brain, and behavior 2003; 2(5): 255-267.
Sharma K, Choi S Y, Zhang Y, Nieland T J, Long S, Li M et al. High-throughput genetic screen for synaptogenic factors: identification of LRP6 as critical for excitatory synapse development. Cell Reports 2013; 5(5): 1330-1341.
Shcheglovitov A, Shcheglovitova O, Yazawa M, Portmann T, Shu R, Sebastiano V et al. SHANK3 and IGF1 restore synaptic deficits in neurons from 22q13 deletion syndrome patients. Nature 2013; 503(7475): 267-271.
Sheng M, Kim E. The Shank family of scaffold proteins. J Cell Sci 2000; 113 (Pt 11): 1851-1856.
Valdivia P, Martin M, LeFew W R, Ross J, Houck K A, Shafer T J. Multi-well microelectrode array recordings detect neuroactivity of ToxCast compounds. Neurotoxicology 2014; 44C: 204-217.
Wagenaar D A, Pine J, Potter S M. An extremely rich repertoire of bursting patterns during the development of cortical cultures. BMC Neurosci 2006; 7: 11.
Wilson H L, Wong A C, Shaw S R, Tse W Y, Stapleton G A, Phelan M C et al. Molecular characterisation of the 22q13 deletion syndrome supports the role of haploinsufficiency of SHANK3/PROSAP2 in the major neurological symptoms. Journal of Medical Genetics 2003; 40(8): 575-584.
Wilson H R, Cowan J D. Excitatory and inhibitory interactions in localized populations of model neurons. Biophysical Journal 1972; 12(1): 1-24.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An in vitro method of identifying a candidate compound for the treatment of a condition associated with neural network dysfunction, the method comprising:
providing an in vitro model of the condition, wherein the model comprises a co-culture comprising a network of inhibitory and excitatory neurons;
detecting a first level of network activity in the model;
contacting the model with a test compound;
detecting a second level of network activity in the model in the presence of the test compound;
comparing the second level of network activity to the first level of network activity; and
selecting a test compound that is associated with an altered level of network activity, as a candidate compound.

2. The method of claim 1, wherein the first level of network activity is a baseline level of network activity, or a level of network activity in the presence of a stimulus that increases activity in the network.

3. An in vitro method of diagnosing the presence of a condition associated with neural network dysfunction in a subject, the method comprising:
providing an in vitro model of the condition, wherein the model comprises a co-culture comprising a network of inhibitory and excitatory neurons, wherein the neurons are obtained by a method comprising differentiating stem cells or neural progenitor cells of a subject suspected of having the condition;
detecting a first level of network activity in the model;
contacting the model with a stimulus that increases activity in the network;
detecting a second level of network activity in the model in the presence of the stimulus;
comparing the second level of network activity to the first level of network activity;
assigning a subject value to the difference between the first and second levels of network activity;
comparing the subject value to a reference value, wherein the reference value represents a level of activity in the presence of the stimulus in a subject who does not have a condition associated with neural network dysfunction; and
identifying a subject as having a condition associated with neural network dysfunction based on the presence of a subject value above the reference value.

4. An in vitro method of selecting a treatment for a condition associated with neural network dysfunction in a subject, the method comprising:
providing an in vitro model of the condition, wherein the model comprises a co-culture comprising a network of inhibitory and excitatory neurons, wherein the neurons are obtained by a method comprising differentiating stem cells or neural progenitor cells of a subject suspected of having the condition;
detecting a baseline level of network activity in the model;
contacting the model with a stimulus that increases activity in the network;
detecting a stimulated level of network activity in the model in the presence of the stimulus;
contacting the model with a test compound;
detecting a baseline level of network activity in the model in the absence of the test compound, and a stimulated level of network activity in the model in the presence of the test compound;
comparing the baseline and stimulated levels of network activity in the presence of the test compound;
assigning a first value to the difference between the baseline and stimulated levels of network activity in the presence of the test compound;
comparing the baseline and stimulated levels of network activity in the absence of the test compound;
assigning a second value to the difference between the first and second levels of network activity in the absence of the test compound;
comparing the first and second values, to detect a level of change in the values;
comparing the change in the values to a reference level of change, wherein the reference level of change represents a level associated with a positive control compound that reduces network activity in the presence of the stimulus; and
selecting a test compound that causes a level of change that is equal to or greater than the reference level of change.

5. The method of claim 2, wherein the stimulus is administration of a chemical agent or an electrical pulse.

6. The method of claim 1, wherein the inhibitory neurons are GABAergic neurons, and the excitatory neurons are glutamatergic neurons.

7. The method of claim 1, wherein the neurons are primary neurons obtained from the brain of an animal.

8. The method of claim 7, wherein the inhibitory neurons are obtained from the striatum of the brain, and/or the excitatory neurons are obtained from the cortex of the brain.

9. The method of claim 1, wherein the neurons are obtained by a method comprising differentiating stem cells or neural progenitor cells or made from cells from a subject having the condition.

10. The method of claim 1, wherein the neurons are obtained by a method comprising differentiating stem cells or neural progenitor cells of a subject having the condition, and the method further comprises administering the selected test compound to the subject.

11. The method of claim 1, wherein detecting a level of network activity comprises detecting one or more of types of bursts, bursting durations, inter-burst interval durations, spike rate within bursts, the number of spikes in each burst; frequency of bursts, and ratios thereof.

12. The method of claim 11, wherein comparing the second level of network activity to the first level of network activity comprises determining the power spectrum for each level, calculating the area under the curve (AUC) in each power spectrum, and comparing the AUC, or comparing the area over a specific frequency range.

13. The method of claim 11, wherein comparing the second level of network activity to the first level of network activity comprises determining the network oscillation by one or both of autocorrelogram or crosscorrelogram analysis for each level, calculating the area under the curve (AUC) in each frequencies, and comparing the AUC, or comparing the area over a specific frequency range.

14. The method of claim 1, wherein the condition associated with neural network dysfunction is autism spectrum disorders, schizophrenia, schizoaffective disorder, depression, obsessive-compulsive spectrum disorders, bipolar disorder, Phelan-McDermid Syndrome (PMS), or epilepsy.

15. A composition comprising a co-culture comprising a network of inhibitory and excitatory neurons, wherein the composition models a neural network dysfunction.

16. The composition of claim 15, wherein the dysfunction is autism spectrum disorders, schizophrenia, schizoaffective disorder, depression, obsessive-compulsive spectrum disorders, bipolar disorder, Phelan-McDermid Syndrome (PMS), or epilepsy.

17. The composition of claim 15, wherein the dysfunction is autism.

18. The composition of claim 15, wherein the inhibitory neurons are GABAergic neurons, and the excitatory neurons are glutamatergic neurons.

19. The composition of claim 15, wherein the neurons are primary neurons obtained from the brain of an animal.

20. The composition of claim 19, wherein the inhibitory neurons are obtained from the striatum of the brain, and/or the excitatory neurons are obtained from the cortex of the brain.

21. The composition of claim 15, wherein the neurons are obtained by a method comprising differentiating stem cells or neural progenitor cells of a subject suspected of having the dysfunction.
